(19) United States
(12) Patent Application Publication
(10) Pub. No.: US 2016/0331794 A1
(43) Pub. Date: Nov. 17, 2016

(54) COMPOSITION FOR USE IN THE TREATMENT OF PERSISTENT COUGH

(71) Applicant: ABOCA S.P.A SOCIETÁ AGRICOLA, Sansepolcro (IT)

(72) Inventors: Valentino MERCATI, Sansepolcro (IT); Laura CAPONE, Sansepolcro (IT); Emiliano GIOVAGNONI, Sansepolcro (IT)

(21) Appl. No.: 15/107,917

(22) PCT Filed: Dec. 22, 2014
(86) PCT No.: PCT/IB2014/067218

§ 371 Date: Jun. 23, 2016

(30) Foreign Application Priority Data
Dec. 23, 2013 (IT) RM2013A000716

(51) Int. Cl. A61K 36/185; A61K 31/715; A61K 31/7004; A61K 36/886; A61K 36/28; A61K 36/53; A61K 36/537; A61K 36/68; A61K 35/644; A61K 31/7016

(52) U.S. Cl. CPC A61K 36/185; A61K 35/644; A61K 31/715; A61K 31/7004; A61K 31/7016; A61K 36/28; A61K 36/53; A61K 36/537; A61K 36/68; A61K 36/886

(57) ABSTRACT

The present application relates to a composition for the treatment of persistent cough, suitable also for pediatric use, processes for the preparation of said composition, and a method for the treatment of persistent cough in adult individuals or in individuals of pediatric age by means of the administration of pharmaceutically effective doses of the above-mentioned composition.

The present application relates to a composition for the treatment of persistent cough, suitable also for paediatric use, processes for the preparation of said composition, and a method for the treatment of persistent cough in adult individuals or in individuals of paediatric age by means of the administration of pharmaceutically effective doses of the above-mentioned composition.

### PRIOR ART

The cough reflex is a primary defense mechanism for the protection of the airways and promotes the removal of material located on the epithelial surface of the respiratory tracts when this exceeds the normal transport ability of the mucociliary system due to quantity, dimensions or rheological characteristics. The cough is the most common cause among adults and children for new admittance into primary care and is often associated with viral respiratory infections of the upper airways (upper respiratory tract infection or URTI). In the case of these pathologies, the symptom may be exaggerated and bothersome, and sometimes can persist even for weeks. This type of cough is caused by a reduction of the stimulus threshold of the reflex, caused by irritants, such as viruses, bacteria, other inhaled substances or unfavourable ambient situations, such as a cold or dry and polluted environment, and in these conditions the ailment can be activated and also maintained by stimuli that normally would be harmless.

One of the most problematic aspects of this type of cough is represented by the persistence of the symptoms, which is a phenomenon present both in adults and in children.

In adults, a cough induced by rhinovirus continues to persist in 20% of subjects on the 10^{th} day. By contrast, in children the cough is present in 70% of children on the 2^{nd} day of illness and is still present in 40% of children on the 9^{th} day of illness (Dicpinigaitis P V, Colice G L, Goolsby M J, Rogg G I, Spector S L, Winther B. (2009). Acute cough: a diagnostic and therapeutic challenge. Cough. 2009 Dec. 16; 5:11). In accordance with that reported by Hay and Wilson (Hay A D, Wilson A, Fahey T, Peters T J. 2003. The duration of acute cough in pre-school children presenting to primary care: a prospective cohort study. Family Practice Vol. 20, No. 6), acute cough in children often lasts a long time, and in fact data from the population shows that both the cough and the nasal secretion may still be present in 50% after one week and in 10-20% after 3 weeks in the case of children having an infection of the respiratory tracts. The subjects in whom the cough persisted beyond the 7-10^{th} day are those predisposed to persistent and chronic cough, even in the absence of a specific organic disease. This can be caused by various independent mechanisms, of which hyperactivity of the respiratory tracts and somatisation of the phenomenon are the most common.

Another problematic aspect of persistent cough is represented by night cough, which is a phenomenon particularly manifest in children, but also occurs in adult subjects.

Very often, a cough manifests itself and is more intense when a person lies down to sleep, disturbing sleep and rest. This phenomenon, as others, is also caused by specific physiopathological mechanisms. Night cough is perhaps the most difficult to resolve and is not well tolerated. Night cough disturbs the sleep of the individual affected and, in the case of a child, disturbs the entire family, creating a feeling of on-going annoyance during the next entire day. The worries of the parents with regard to persistent cough may reach extreme levels of intensity (fear that the child will suffocate, pulmonary damage, infectious complications of the lower airways, etc.). Chang, in Chang A B. Cough. 2005, asserts that “ . . . the burden of coughs in children and in families has not yet been described accurately”.

Both the persistence and the greater intensity of the cough, including of night cough, are caused by some of the physiopathogenic aspects that induce the cough. The following have a primary role: mediators of inflammation, intense mucous secretions and possibly post-nasal drip. Mediators of inflammation act both locally, stimulating the epipharyngeal nerve endings and increasing the sensitivity of the cough receptors, and haematically, unleashing inappropriate tussigenic reflexes. The greater is the local irritation caused by the mediators, the higher is the risk of persistence of the cough. The mucous secretions produced to protect the mucosa from the irritation caused by the pathogens may be so abundant and viscous that they become stagnant in particularly sensitive areas and create abnormal sensory and/or mechanical stimulations causing what is known as post-nasal drip (PNDS, post-nasal drip syndrome). This is an irritative tussigenic stimulus of mechanical type linked with the presence of mucus, often in the form of viscous fibres which are difficult to expel from the pharynx and which, descending far into the pharynx, cause persistent mechanical stimulation of the upper tract of the larynx, resulting in an on-going and irritating cough. The zones affected by this phenomenon include the rear wall of the pharynx, of which the plexus of glossopharyngeal and vagus nerve endings is exposed to the irritant action of the flows of mucus that arrive from the pharynx, and this can unleash tussigenic reflexes aimed at the expulsion of the mucus perceived as a foreign body. The on-going mechanical stimulus results in an annoying need to cough, and the cough contributes to inflammation and irritation of the mucosa, thus creating a vicious circle that promotes the self-maintenance of the symptom and persistence thereof. In practice, the cough persists by widespread pharyngeal inflammation, by peeling of the epithelial cells, and damage of the nerve endings, which makes them hyperreactive.

The release of inflammation mediators by the mucosa of the higher airways can increase the sensitivity of the efferent nerves, causing hyperactivity of the upper airways, which has been demonstrated in 40% of subjects affected by persistent-chronic cough not caused by an organic respiratory disease. The hyperreactivity of the upper airways may be an important mechanism in the induction of a cough tending to persist completely independently of a pathology of the lower airways (Carney I K, Gibson P G, Murree-Allen K, Saltos N, Olson L G, Hensley M J. A systematic evaluation of mechanisms in chronic cough. Am J Respir Crit Care Med. 1997 July; 156(1):211-6).

Oedema and constriction of the pharynx seem to be involved significantly in this phenomenon. Inflammatory pharyngeal damage may promote the access of irritant substances to the nerve endings of the submucosa, thus activating constriction reflexes and also activating pharyngeal/bronchial reflexes.

It is therefore clear that, in order to avoid the persistence of the cough beyond 3 weeks, it is necessary not only to act on the disease causing the cough, but also to prevent and obtain a desensitisation of the nerve endings.

It can be stated without a shadow of a doubt that there is currently no effective treatment for persistent cough.

The available treatments are constituted by antihistamines, which are central antitussives, such as codeine and dextromethorphan, cough sedatives with peripheral action, which are usually mild local analgesics/anesthetics, such as dropropizine, cortisones via inhalation or systemic route, and mucolytic drugs, which exhibit a fluidizing effect, by means of which they reduce the viscosity of the mucus, promoting expulsion, such as ambroxol and carbocysteine or acetylcysteine.

The objective of this type of therapy is to reduce the intensity and frequency of the cough in the short term (Bolser D C. Cough suppressant and pharmacologic protussive therapy: ACCP evidence-based clinical practice guidelines. Chest. 2006 January; 129(1 Suppl):238S-249S). None of these drugs seems to be actually effective on persistent cough, except perhaps for codeine, which, however, has significant use limitations given that it can cause respiratory depression, nausea, stypsis and dependence (Chung in Redington A E, Morice H. 2005. Acute and chronic cough. Taylor & Francis). The efficacy of inhaled cortisones also seems to be inconsistent in adults and in children according to two very recent revisions of the Cochrane Library (Johnstone K J, Chang A B, Fong K M, Bowman R V, Yang I A. Inhaled corticosteroids for subacute and chronic cough in adults; Cochrane Database Syst Rev. 2013 Mar. 28; 3:CD009305. Review; Anderson-James S, Marchant J M, Acworth J P, Turner C, Chang A B Inhaled corticosteroids for subacute cough in children Cochrane Database Syst Rev. 2013 Feb. 28; 2:CD008888) in spite of the wide use made of these in clinical practice. The search for a therapeutic solution to the cough accompanying persistent cough has been studied by many research centres dedicated to coughs, but for now does not show great prospects for success.

In the literature (PubMed database) it is not possible to find any works that confirm the efficacy and thus the usefulness of sedative and mucolytic drugs for persistent cough (Testagrossa O, Siclari E. 2005. Farmaci contro la tosse? Non sono efficaci. Quaderni acp; 12(4): 178-181). In fact, significant side effects have been pointed out for some of these drugs, such as for mucolytics, for which there is a ban on prescriptions for children less than 2 years of age. Dextromethorphan, one of the most frequently used drugs, seems to be only marginally better than the placebo in the treatment of coughs (Lee P. C. L, Jawad M. S. M, Eccles R. 2000. Antitussive efficacy of dextromethorphan in cough associated with acute respiratory tract infection. J Pharm Pharmacol. 52:1137-1142). Neither dextromethorphan nor diphenhydramine have been found to be better than the placebo in diminishing night cough in children (Yoder K E, Shaffer M L, La Tournous S J, Paul I M.). 2006. Child assessment of dextromethorphan, diphenhydramine, and placebo for nocturnal cough due to upper respiratory infection. Clin Pediatr (Phila). 45(7):633-40).

It is true that in many studies a single serial dose of honey has demonstrated an ability to soothe the intensity of acute cough, even compared with dextromethorphan. However, all studies have involved children suffering from acute cough only for seven days or less. No study has confirmed the efficacy of honey, either as a single component or in combination with other natural substances, in coughs persisting beyond seven days (night and/or day), which have a much higher likelihood also of persisting beyond 3 weeks.

It is therefore clear that finding a solution to persistent cough would represent an important step forwards in resolving one of the problems that more frequently affects the adult and paediatric population and that tends to persist in the long term in a significant proportion of the population in question.

### SUMMARY OF THE INVENTION

The fact that no effective solution has yet been found after so many years of research leads us to rethink the problem on the basis of an accurate analysis of the physiopathological phenomena at the root thereof. In this sense, at least for some of the subjects affected, inflammatory phenomena affecting the upper airways seem to merit particular attention, in particular inflammation of the pharyngeal mucosa. This would appear to have a significant role in the triggering and maintenance of the phenomenon of persistence, given that it has been documented that inflammatory pharyngeal damage can promote the access of irritant substance to the nerve endings of the submucosa, activating constriction reflexes and pharyngeal/bronchial reflexes.

In this sense, the protection of the mucosa against contact with irritant agents and against irritative action thereof and defense against the mechanical-irritative stimulus of the mucus represent an innovative way of approaching the problem that is highly different from the interventions implemented previously, which for the most part tend to block the cough reflex at central and peripheral level or tend to modify the consistency of the mucus and have never been aimed at intervening at the root of the phenomenon, that is to say protecting pharyngeal mucosa against contact with external agents, thus hindering the inflammatory process caused as a result and consequently preventing hypersensitisation of the nerve endings.

The fact that certain subjects may also have the need to cough, moreover intensely, due to stimuli at the rear wall of the pharynx seems to be confirmed also by anatomical knowledge, which is rather recent, and by studies that have investigated whether the cough also can be stimulated or modified by means of mechanical, gustatory and thermal stimuli applied in particular sensorial areas. One of these areas would be represented by the plexus of vagus and glossopharyngeal sensorial nerve endings located in the rear wall of the oropharynx. Mu and colleagues (Mu L, Sanders I. (2000). Sensory nerve supply of the human oro- and laryngopharynx: a preliminary study. Anat Rec. 2000 Apr. 1; 258(4):406-20) have demonstrated that the pharyngeal branch of the glossopharyngeal nerve and the vagus nerve provide sensorial innervations at the rear and lateral walls of the oropharynx and at the pillars of the tonsils. This pharyngeal plexus of nerve fibres, if stimulated, can provoke a gag or cough reflex as has been demonstrated by an interesting study by Wheeler-Hegland (Wheeler-Hegland K, Pitts T, Davenport P W. (2010) Cortical gating of oropharyngeal sensory stimuli. Front Physiol. 2010 Jan. 25; 1:167).

The authors have in fact observed that a slight puff of air directed towards the rear part of the oropharynx, of such an intensity that would not cause a sensation of pressure, but only delicate stimulus, can trigger an intense need to cough or, in some cases, a coughing fit.

Also according to Madison and Irwin (Madison J M, Irwin R S. Pharmacotherapy of chronic cough in adults. Expert Opin Pharmacother 2003; 4:1039-1048), mechanical stimuli on the upper airways can cause coughing in conditions of hyperactivity of the airways caused by URTI.

In light of all this knowledge, the authors therefore believe that a persistent inflammatory process at the pharynx may constitute the cause of an acute cough that may have a greater likelihood of persisting as a symptom, the greater the quantity of irritant agents free to come into contact with the mucosa and to cause production of inflammation mediators and the longer the length of time the mucus is left to become dense and viscous so as to thus act as a mechanical-irritative stimulus, and that interrupting the vicious circle initiated by the irritation of the mucosa, the unleashed responses by inflammation mediators derived therefrom, and the mucus itself would represent the keystone of effective treatment of this problem.

The authors therefore believe that intervening in an effective and innovative manner in the phenomenon of a cough does not so much require a soothing of the manifestation, as has been sought for many years, but requires the cause of the phenomenon to be tackled, limiting the damage caused by the contact of the virus and of other irritant agents with the mucosa and by the irritant action thereof, which triggers the production of inflammation mediators and mucus.

The authors have therefore attempted to obtain an indirect anti-inflammatory action obtained not on account of pharmacological actions, but with an exclusive mechanical-physical protection process.

The efficacy of a composition comprising, as active ingredient, a mixture consisting of:

polysaccharides of plant origin between 0.06 and 5.5% by weight of said composition;
antioxidants of plant and/or natural origin between 0.005 and 1.2% by weight of said composition;
resins of plant and/or natural origin between 0.008 and 1.5% by weight of said composition;
saponins of plant origin between 0.0002 and 0.03% by weight of said composition;
honey between 2.5 and 85% by weight of said composition and
further glucose, sucrose, fructose and/or inulin between 0 and 87% by weight of said composition was tested in the treatment of persistent cough. The classes of functional components originating from medicinal plants and natural substances were selected for the indirect mechanical-physical and anti-inflammatory effects that they are able to implement locally. The experimental data and double-blind clinical tests carried out by the inventors demonstrated that the combination of the above-mentioned active ingredients is able to guarantee effective protection of the mucosa against damage linked to contact of said mucosa with external irritant agents and is also able to thus limit the production and penetration into the submucosa of inflammation mediators and to provide protection against the continuous irritative stimulus of the mucus fibres with an indirect anti-inflammatory and antioxidant effect, and that the composition is therefore effective for use in the treatment of persistent cough, which includes, for example, cough associated with UTRI and/or post-nasal drip.

The fact that this is a real innovation is also demonstrated by the fact that the respected medical scientific literature (Bruton I., Chabner B. A., Knollmann B. C. GOODMAN & GILMAN LE BASI FARMACOLOGICHE DELLA TERAPIA. Zanichelli, 2012, 12^{th} edition; Pratter M R. Cough and the common cold: ACCP evidence-based clinical practice guidelines. Chest. 2006 January; 129(1 Suppl):72S-74S) does not appear to contain any reference to a therapy of persistent cough by means of a mechanism of action similar to that of the composition of the invention, nor to the use of a similar composition for use in the treatment of persistent cough, that is to say coughs characterised by a duration of more than a week or more than 10 days. The present application thus relates to a composition for use in the treatment of persistent cough comprising, as active ingredient, a mixture consisting of:

polysaccharides of plant origin between 0.06 and 5.5% by weight of said composition;
antioxidants of plant and/or natural origin between 0.005 and 1.2% by weight of said composition;
resins of plant and/or natural origin between 0.008 and 1.5% by weight of said composition;
saponins of plant origin between 0.0002 and 0.03% by weight of said composition;
honey between 2.5 and 85% by weight of said composition and
further glucose, sucrose, fructose and/or inulin between 0 and 87% by weight of said composition for use in the treatment of persistent cough; a process for preparing said composition, and a method for treatment of persistent cough comprising the administration of said composition in pharmacologically effective doses to an individual in need thereof.

### DETAILED DESCRIPTION OF THE FIGURES

FIG. 1, taken from Shields 2007, shows the variation of cough patterns over time; as can be seen, persistent cough demonstrates an increase over time without remission of the symptoms. Whereas acute cough, even with delayed recovery, starts to decrease from the first week, persistent cough increases constantly over time, even for periods longer than a month.

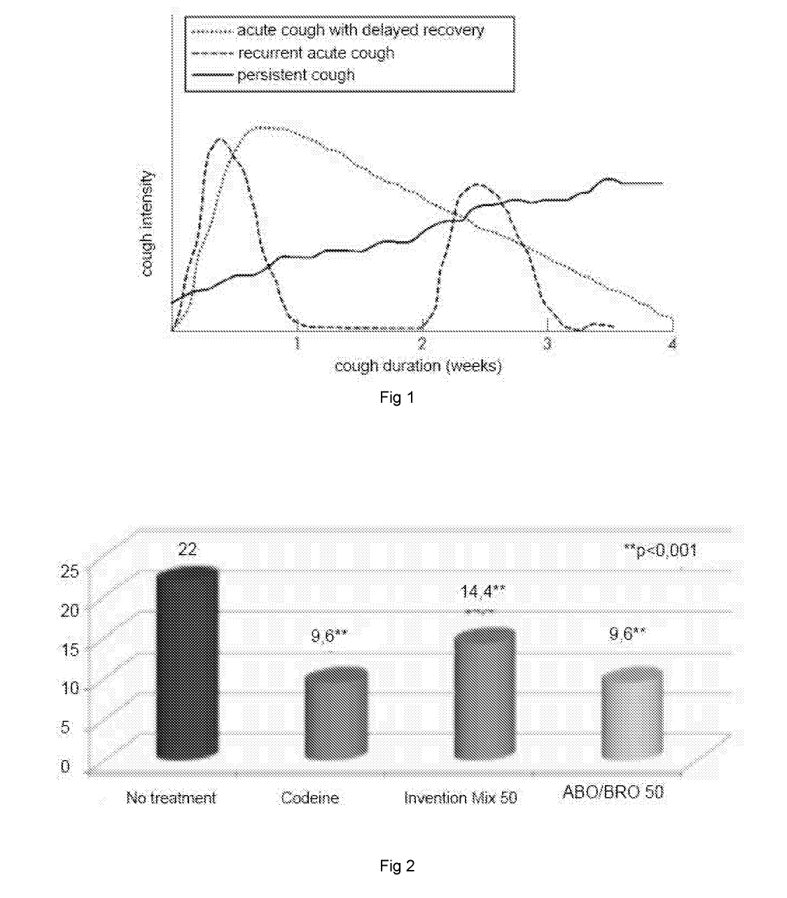

FIG. 2 shows the pre-clinical experiment on coughing following acute irritation with capsaicin. The coughing fits following acute irritation with capsaicin as described in experimental example 1 were measured.

The application of the experimental products 30 minutes prior to induction of the cough with capsaicin (45 μM, 30 μg/ml in 10% ethanol in saline solution for 3 minutes) leads to a significant decrease of coughing fits measured 1 minute following the end of the stimulus in the three cases: positive (codeine), mixture of the invention (50 μl), and commercial product based on dextromethorphan indicated by the abbreviation ABO/BRO.
The graph shows significant efficacy with p<0.001 for all three products.

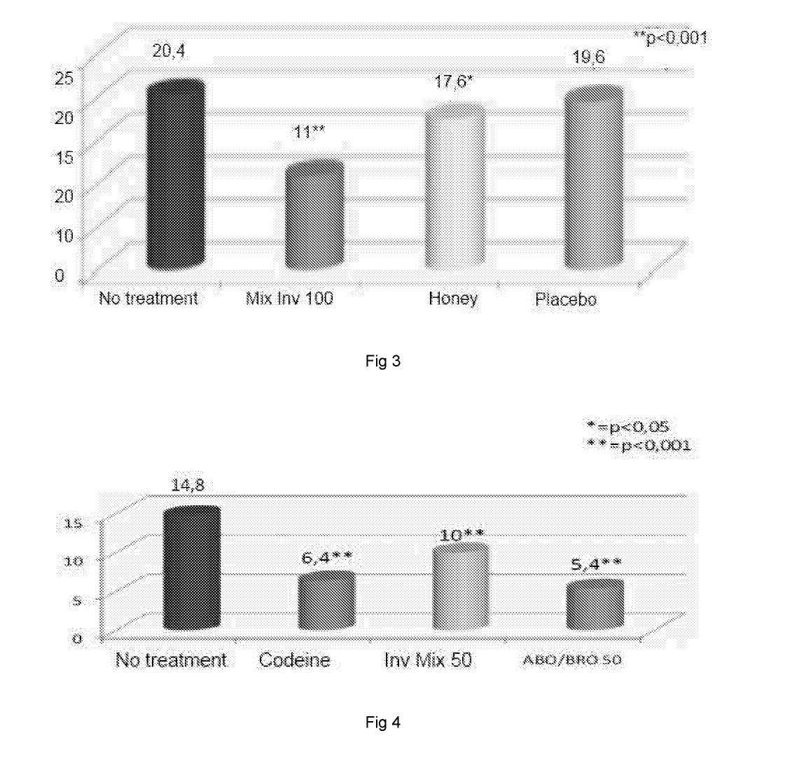

FIG. 3 (experimental example 1) shows the same experiment shown in FIG. 2, in which, however, values for the negative control, the mixture according to the invention (100 μl), honey and the placebo are taken into consideration.

In this case, the efficacy is much more significant for the mixture of the invention compared to just honey, whereas the placebo has no effect at all.

FIG. 4 (experimental example 1) shows the pre-clinical experiment on coughing following acute irritation with capsaicin. The coughing fits following acute irritation with capsaicin as described in experimental example 1 were measured.

The application of the experimental products 30 minutes prior to induction of the cough with capsaicin (45 μM, 30 μg/ml in 10% ethanol in saline solution for 3 minutes) leads to a significant decrease of coughing fits measured 30 minutes following the end of the stimulus in the three cases: positive control (codeine), mixture of the invention (50 μl), and commercial product based on dextromethorphan indicated by the abbreviation ABO/BRO.
The graph shows significant efficacy with p<0.001 for all three products.

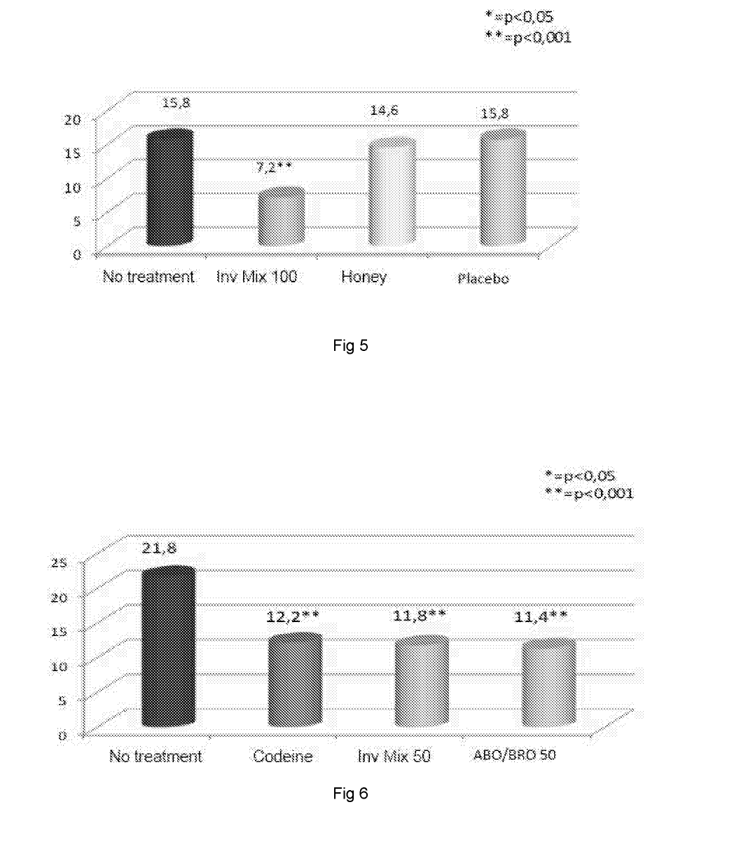

FIG. 5 (experimental example 1) shows the same experiment shown in FIG. 4, in which, however the values for the negative control, the mixture according to the invention (100 μl), honey and the placebo are taken into consideration.

Whereas honey loses its efficacy over time, the mixture of the invention maintains high significance (p<0.001) even over time.

FIG. 6 (experimental example 1) shows the pre-clinical experiment on coughing following chronic irritation with capsaicin. The coughing fits following chronic irritation with capsaicin as described in experimental example 1 were measured.

The application of the experimental products 4 hours prior to induction of the cough with capsaicin (45 μM, 30 μg/ml in 10% ethanol in saline solution for 3 minutes) leads to a significant decrease of coughing fits measured 1 minute following the end of the stimulus in the three cases: positive control (codeine), mixture of the invention (50 μl), and commercial product based on dextromethorphan indicated by the abbreviation ABO/BRO.

The graph shows significant efficacy with p<0.001 for all three products.

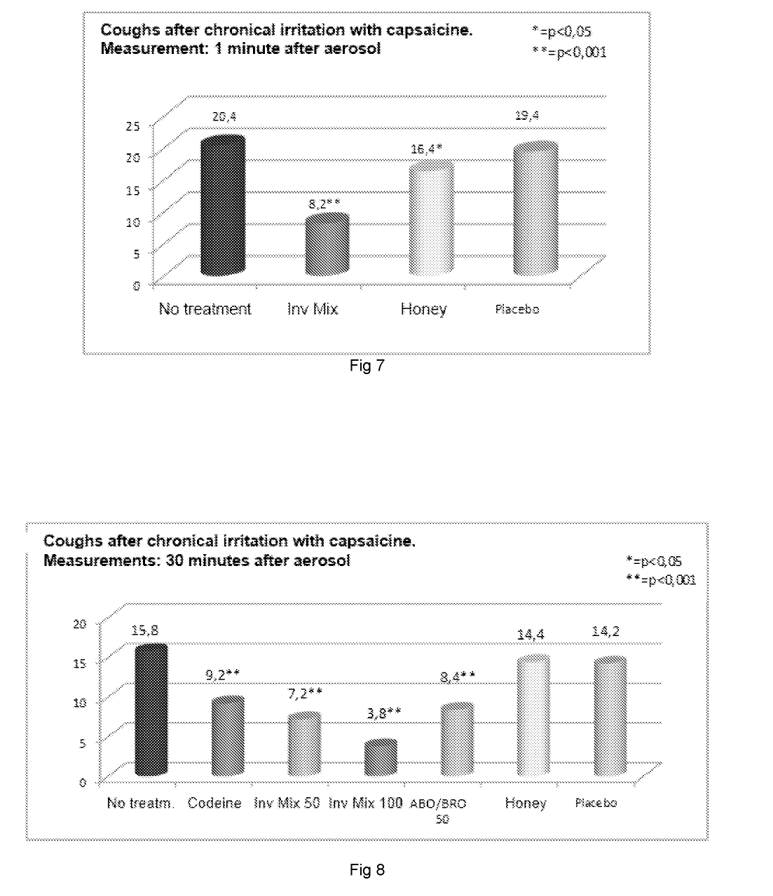

FIG. 7 (experimental example 1) shows the same experiment shown in FIG. 4, in which, however, the values for the negative control, the mixture according to the invention (100 μl), honey and the placebo are taken into consideration.

Whereas honey loses its efficacy over time, the mixture of the invention maintains high significance (p<0.001) even over time.

FIG. 8 (experimental example 1) shows the response after 30 minutes from capsaicin in chronic treatment, the experiment showing a trend similar to acute treatment: the cough decreases physiologically, honey loses efficacy, the products having central action substantially maintain the efficacy observed 30 minutes before, the mixture of the invention has improved performance, probably due to the basic protection of the mucosa, which allows it to positively modulate its sensitivity to the irritant stimuli. In this case, a greater efficacy of higher doses is observed, probably due to a greater presence of the product over time on the mucosa, which is thus able to implement a greater protective effect.

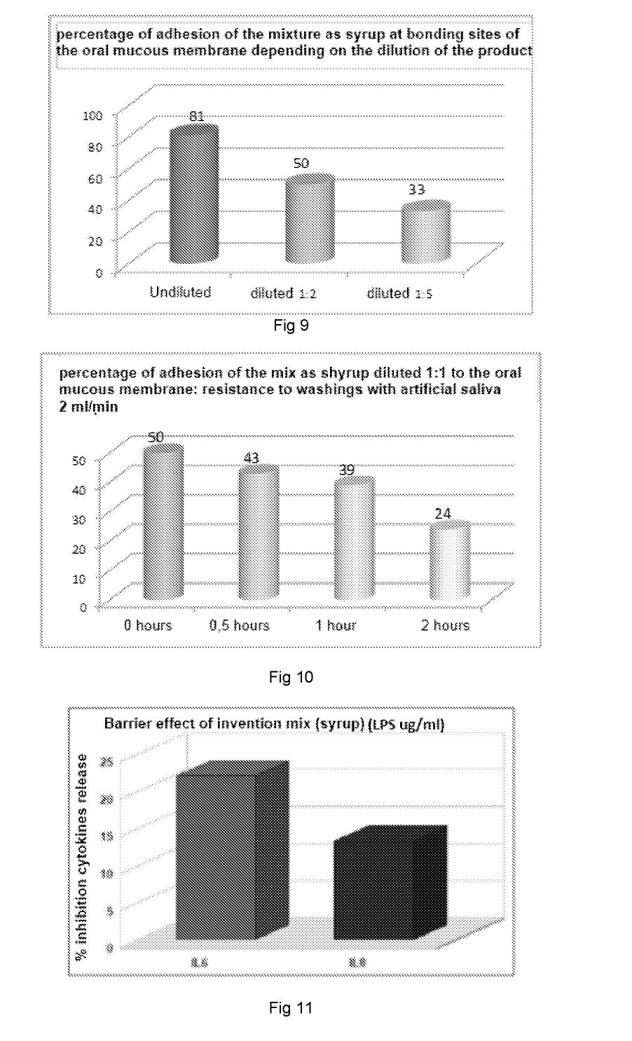

FIG. 9 (experimental example 2) shows the mucoadhesion of the mixture of the invention to oral mucous membrane at various dilutions, the measurement showing the percentage of adhesion of the mixture in liquid form (syrup) at bonding sites of the oral mucous membrane depending on the dilution of the product.

FIG. 10 (experimental example 2) shows the resistance of the mucoadhesion to washing with simulated saliva.

The graph shows the percentage of adhesion of the mixture of the invention in liquid form (syrup) diluted 1:2 at bonding sites of the oral mucous membrane: resistance to washing with artificial saliva 2 ml/min for a period up to 2 hours.

FIG. 11 shows the protective effect of the mixture by means of formation of a barrier film measured in the experiment described in experimental example 3.

### GLOSSARY

PERSISTENT COUGH: in accordance with the present invention, persistent cough is a cough of which the symptoms, without improvement thereof, persist for a duration longer than a week, ten days, two weeks or even three weeks.

RESINS: the definition of resins in accordance with the present description corresponds to that known in the literature for resins of plant origin, that is to say ‘resins’ means a complex group of solid substances, occasionally liquid, that are insoluble in water, but soluble in alcohol, ethers and chloroform. Resins are usually produced by plants as a response to a trauma or stress (injury, pathogenic attack). Resins could be derived from the transformation of parietal polysaccharides and even from starch. It is hypothesized that they have the role of defending the plant from insects, fungi or other infections, or of healing wounds. In combination with essential oils and gums, resins form oleo-resins and gum-resins respectively (or oleo-gum-resins). Combined with aromatic acids, they form balsamic or balsam resins. The resin fraction is composed primarily by triterpenes or by diterpenes in the case of resins from Coniferae.

SAPONINS: Also for saponins, the definition known in the literature for saponins of plant origin is suitable. In accordance with the invention, triterpene saponins are therefore largely widespread in the plant kingdom, defend plants against attack from bacteria and fungi, and are glycosides of natural origin, from the acid hydrolysis of which they release sugars and aglycones (referred to as sapogenins).

This class of compounds is known for its amphoteric characteristics (lipophilic portion and hydrophilic portion): in water the molecules align themselves with one another, orienting the lipophilic part towards the surface and reducing the surface tension.

Following oral administration, saponins tend to be absorbed only minimally, and this on the one hand allows them to develop their mechanical action at the site of application and on the other hand to minimise their toxicity.

POLYSACCHARIDES OF PLANT OR MUCILAGINOUS ORIGIN: These are polysaccharide macromolecules which, upon contact with water, form colloidal solutions or gels. They can also define plant hydrocolloids. They are normally formed of plant cells. They are preserved in predefined histological structures. They are able to absorb a large quantity of water, generating fluid gels (hydrocolloids).

Mucoadhesion of polysaccharides of plant origin has been known for 40 years and, compared with other synthetic polymers, has improved bonding power and also adhesion time, it also is not influenced as much by pH.

The difference between time of mucoadhesion of synthetic polysaccharides and natural polysaccharides is considerable. For example, in some standardised in vitro tests, regular chitosan has an adhesivity of approximately 50 hours, whereas the natural polysaccharides not developed synthetically adhere within a few hours.

SUGARS: In accordance with the present invention, the term “sugars” means sucrose, glucose and fructose. A high sugar concentration confers adhesive properties to a product and increases the viscosity-consistency thereof, providing it with improved adhesion at the site of application.

The sweet taste involves an increase in salivation and an increase of the production of mucus in the airways. Sugars also have the property of inducing salivation and of detaining water, acting as humectants with a direct emollient effect when they come into contact with superficial epithelia.

ANTIOXIDANTS: Antioxidant agents or antioxidants according to the invention are antioxidant substances of plant origin, represented for the majority by polyphenols that include, for example, phenols, tannins, and flavonoids, and by vitamins, which may have a protective role in respect of oxidative damage. These antioxidant agents can act autonomously or in a manner in which they interact positively with one another, thus exerting reciprocal protection in respect of free radicals. The most commonly known natural antioxidant compounds are polyphenols, of which flavonoids represent the most important sub-class. Oxidation involves an irritation and an increase of the irritability of the mucosa, thus giving way to a chain of complex inflammatory systems that can be avoided or reduced by preventing the initial reaction in a non-immunological or pharmacological manner.

ESSENTIAL OILS: Essential oils mean those oils that are obtained from plant material using extraction procedures that make it possible to concentrate the volatile chemical compounds contained in the plant. The extraction of the essence from the plant material can be implemented by distillation in vapour stream, by dry distillation, or by means of mechanical cold-pressing processes. During the phase of distillation, the natural essence produced from the plant is concentrated up to 100 times or more. The essences are produced in a particular way from some families of plants, such as Pinaceae, Myrtaceae, Labiateae, Geraniaceae, Umbelliferae, etc. The essence plants are commonly used, and the essential oils obtained by distillation have been known from ancient times to experts in the sector. These are mixtures of organic substances, volatile for the most part, with characteristic aromatic fragrance, which is rather prominent in general.

Some essential oils (orange, lemon, mint, etc.) can be used for aromatizing purpose on account of their pleasant taste, and confer to the product containing them a sensation of freshness by the instantaneous evaporation of the volatile substances at the moment of application.

### DETAILED DESCRIPTION OF THE INVENTION

The present description thus concerns a composition comprising, as active ingredient, a mixture consisting of:

polysaccharides of plant origin between 0.06 and 5.5% by weight of said composition;
antioxidants of plant and/or natural origin between 0.005 and 1.2% by weight of said composition;
resins of plant and/or natural origin between 0.008 and 1.5% by weight of said composition;
saponins of plant origin between 0.0002 and 0.03% by weight of said composition;
honey between 2.5 and 85% by weight of said composition and
further glucose, sucrose, fructose and/or inulin between 0 and 87% by weight of said composition for use in the treatment of persistent cough.
The expression “further glucose, sucrose, fructose and/or inulin” indicates clearly that the formulation provides for the presence of honey in certain proportions that can be combined with other sugars, which may be glucose, sucrose, fructose or a mixture thereof, the phrase thus being suitable for substitution by the phrase “additionally also glucose, sucrose, fructose and/or inulin” or by the phrase “additionally glucose, sucrose, fructose and/or inulin”.
For the purposes of the present invention, persistent cough is defined as in the literature, that is to say a type of cough that lasts for longer than a week to ten days. In particular, persistent cough is a cough in which the symptoms do not reduce after a week to ten days, in fact in many cases the persistent cough can have a duration, normally associated with an increase of the symptoms, of greater than ten days, or even two weeks or even three weeks.
The experimental data provided below shows that the composition according to the present invention is suitable for the treatment of the typology of persistent cough, that is to say a cough that lasts for longer than a week to ten days, two weeks or even more than three weeks.
As explained in the prior art section above, persistent cough can present itself in combination with upper respiratory tract infection (URTI), such as infections of a viral type, or can also present itself in combination with intense mucous secretions and post-nasal drip.
Such cough typology is present in all age ranges and must be treated particularly delicately in paediatric or geriatric age or even during pregnancy, insofar as current therapies suggest the use of cortisones, antitussives and/or mucolytics that often are not advisable in more delicate patient groups and that in any case have been found to be less effective for the treatment of this type of cough.
For example, mucolytic drugs increase the fluidity of the mucus using mechanisms of depolymerization of the mucoprotein complexes and of the nucleic acids. They slice the polymers of mucin, breaking the disulphur bonds and reducing the viscoelasticity of the mucus.
In 2010, the French Drug Agency withdrew the license for use below two years of age for carbocysteine and acetylcysteine because use thereof was associated with paradoxic bronchorrhea and acute respiratory distress during the course of respiratory infections. The French pharmacovigilance system evidenced, in practice, a rise in cases of respiratory obstruction and worsening of respiratory pathologies in children less than 2 years of age treated with mucolytics (exactly the opposite of that which it is sought to obtain with these drugs).
In November of 2010, with a similar measure, the Italian Medicines Agency (AIFA) decreed a ban on use below 2 years of age of the active ingredients: acetylcysteine, carbocysteine, ambroxol, bromhexine, sobrerol, neltenexine, erdosteine and telmesteine.
Sedative drugs with central action (derived from opiates) inhibit the activity of the centre of the cough with risk of respiratory depression in children, for which reason they are always contraindicated in those below 2 years of age.
Non-opiate sedative drugs can have peripheral or mixed effect depending on their ability to surpass the blood-brain barrier. One example is dropropizine, which has both central and peripheral action, displaced from its levorotatory isomer, levodropropizine, which has only peripheral action. Sedatives with peripheral action act by raising the threshold of excitability of the receptors arranged on the mucosa of the respiratory tracts, thus tending to eliminate the symptom by also annulling the physiological activity of said receptors of clearance of secretions. These drugs, although also indicated below one year of age, are not prescribed unless in critical situations, such as in the rarest cases of whooping cough.
The composition according to the invention, given the components thereof of plant and natural origin, is instead suitable for treatment that can be carried out on adult patients, in patients of adolescent age, on patients of geriatric age or on patients of paediatric age.
In particular, one double-blind clinical study reported in the experimental section, carried out on patients of paediatric age, demonstrates the efficacy of the composition of the invention for the treatment of persistent cough in children.
The above-described components present in the mixture of active ingredients of the composition according to the invention are of plant or natural origin, as already mentioned.
All the embodiments described above apply, individually, to each of the composition examples described below.
In one embodiment of the invention, the antioxidants can be polyphenols, in particular phenols, tannins, flavonoids or a mixture thereof, or also vitamins (alone or combined with polyphenols) present in the mixture and are derived from one or more of the following plants: Helychrisum italicum, Thymus vulgaris, Thymus serpyllum, Sambucus Nigra, Marrubium vulgare, Tilia cordata, Tilia platophyillos, Verbascum densiflorum, Glycyrrhiza glabra, Salvia officinalis, Echinacea purpurea, Plantago lanceolata, Grindelia robusta and/or from propolis.

In another embodiment of the invention, the polysaccharides are derived from one or more of the following plants: Plantago lanceolata, Aloe vera, Althaea officinalis, Malva syslvestris, Cetraria islandica, Verbascum densiflorum, Glycyrrhiza glabra, Tilia cordata, Tilia platophyillos, Echinacea pallida, Echinacea purpurea, Echinacea angustufolia, Helychrisum italicum, Salvia officinalis, Grindelia robusta.

In a further embodiment of the invention, the resins of the above-described mixture are derived from one or more of: Grindelia robusta, Commiphora myrrha, Boswelia serrata, Pinus mugo, Pinus sylvestris, propolis.
In yet a further embodiment, the saponins are derived from one or more of: Grindelia robusta, Verbascum densiflorum, Glycyrrhiza glabra, Marrubium vulgare, Poligala senega, Plantago lanceolata.
Lastly, the honey present in the mixture can be bee honey and/or honeydew.
In a preferred embodiment, the antioxidants, polysaccharides, resins and saponins are derived from the sources described above.
By way of example, the antioxidants can be polyphenols, in particular phenols, tannins, flavonoids or a mixture thereof, or also vitamins (alone or combined with polyphenols) and can be derived from one or more of Helychrisum italicum, Grindelia robusta, Glycyrrhiza glabra, Plantago lanceolata, Thymus vulgaris, Sambucus Nigra; the polysaccharides can be derived from one or more of Helychrisum italicum, Grindelia robusta, Glycyrrhiza glabra, Plantago lanceolata, Echinacea pallida, Echinacea purpurea, Echinacea angustufolia, Thymus vulgaris; the resins can be derived from one or more of Grindelia robusta, Commiphota myrrha, Boswelia serrata and the saponins can be derived from one or more of: Grindelia robusta, Verbascum densiflorum, Glycyrrhiza glabra.
In accordance with a further embodiment, the antioxidants can be polyphenols, in particular phenols, tannins, flavonoids or a mixture thereof, or also vitamins (alone or combined with polyphenols) and can be derived from Helychrisum italicum, Grindelia robusta, Glycyrrhiza glabra, Plantago lanceolata; the polysaccharides can be derived from Helychrisum italicum, Grindelia robusta, Plantago lanceolata; the resins can be derived from one or more of Grindelia robusta, and the saponins can be derived from one of more of: Grindelia robusta and/or Glycyrrhiza glabra.
The above-mentioned classes of substances that form part of the mixture of active ingredients can be present in the composition in the form of fractions of plant extracts or in the form of non-fractionated plant extracts, such as hydroalcohol extracts optionally lyophilised or mixtures thereof.
The fractions of the plant extracts containing the classes of substances of interest can be obtained using techniques known to a person skilled in the art as fractionated precipitation in hydroalcoholic solvents, filtration, ultrafiltration, nanofiltration, passing through resin, or mixed methods. The following are noted by way of example: fractionation of the phenol substances by passing it through adsorbent resin; fractionated precipitation by means of alcohol in order to obtain fractions enriched with polysaccharides, or by means of water to obtain fractions enriched with resins.

For grindelia, it is possible for example to use lyophilised hydroalcoholic extracts of flowering and/or non-flowering tops, that is to say end areas of the plant that contain stem leaves (understood as branches and not as the main stem of the plant) and flowers of grindelia or at least one of these components or, as mentioned above, fractions of such extracts in which one or more of the classes of above-mentioned compounds are present (polyphenols, polysaccharides, resins, saponins). For helichrysum, it is possible in a similar manner to use hydroalcoholic extracts of flowering and/or non-flowering tops, such extracts possibly being lyophilised. In one embodiment of the invention, it is possible to use fractions of such extracts that contain one or more of the above-mentioned classes of substances.

With regard to ribwort plantain, the extract can be a hydroalcoholic extract or fractions thereof containing one or more of the above-mentioned classes of substances of any part of the plant, such as fresh and/or dried leaves. In the case of thyme, it is possible to use a hydroalcoholic extract or fractions thereof as described above of any part of the plant, such as fresh and/or dried leaves and/or flowering tops. For elder, it is possible to use a hydroalcoholic extract or fractions thereof as described above of any part of the plant, such as fresh and/or dried berries. For horehound, it is possible to use a hydroalcoholic extract or fractions thereof as described above of any part of the plant, such as fresh and/or dried leaves and/or flowering tops. For mullein, it is possible to use a hydroalcoholic extract or fractions thereof as described above of any part of the plant, such as fresh and/or dried leaves and/or flowering tops and/or flowers. For liquorice it is possible to use a hydroalcoholic extract or fractions thereof as described above of fresh and/or dried roots. For lime it is possible to use a hydroalcoholic extract or fractions thereof as described above of fresh and/or dried leaves and/or flowers and/or bracts. For sage, it is possible to use a hydroalcoholic extract or fractions thereof as described above of any part of the plant, such as fresh and/or dried leaves and/or flowering tops. For Echinacea it is possible to use a hydroalcoholic extract or fractions thereof as described above of any part of the plant, such as fresh and/or dried leaves and/or flowering tops and/or roots. For aloe it is possible to use a hydroalcoholic extract or fractions thereof as described above of aloe gel (the gel is found in the leaves of aloe). For althea it is possible to use a hydroalcoholic extract or fractions thereof as described above of fresh and/or dried roots. For mallow it is possible to use a hydroalcoholic extract or fractions thereof as described above of any part of the plant, such as fresh and/or dried leaves and/or flowering tops. For lichen it is possible to use a hydroalcoholic extract or fractions thereof as described above of any part of the plant, such as the fresh and/or dried thallus. For pine it is possible to use a hydroalcoholic extract or fractions thereof as described above of fresh and/or dried shoots and/or buds and/or young cones and/or needles. For polygala it is possible to use a hydroalcoholic extract or fractions thereof as described above of any part of the plant, such as the fresh and/or dried root. For frankincense it is possible to use a hydroalcoholic extract or fractions thereof as described above of the dried unrefined oleo-gum-resin. For propolis it is possible to use a hydroalcoholic extract or fractions thereof as described above of the unrefined oleo-resin. For myrrh it is possible to use a hydroalcoholic extract or fractions thereof as described above of dried unrefined oleo-gum-resin.
In accordance with the invention, the honey, as already mentioned, can be bee honey, honeydew or a combination thereof.
Bee honey can be a wildflower honey, an acacia honey, a lime honey, an orange flower honey, a chestnut honey acacia honey (Robinia pseudoacacia)
Citrus honey
Tree-of-heaven honey
Carob tree honey (Ceratonia siliqua)
Chestnut honey (Castanea sativa)
Rapeseed honey (Brassica napus)
Corbezzolo honey (Arbutus unedo) (has the property of being bitter)
Marasca cherry honey (Prunus mahaleb)
Ivy honey
Heather honey (Erica spp.)
Eucalyptus honey
Sunflower honey
Lavender honey
Leguminous honey:
   clover
   alfalfa
   birdsfoot trefoil
   melilotus officinalis
   sulla
Honeydew honey:
   fir honeydew honey
   Metcalfa pruinosa honey dew honey, a citrus flatid planthopper of American origin
   hazelnut honeydew honey
   oak honeydew honey
   apple tree honey
Rhododendron honey
Dandelion honey
Lime honey
Thyme honey
Cardoon honey
Asphodel honey
Orange blossom honey
Bergamot honey
Rosemary honey
Blackberry honey
Sainfoin honey
Manuka honey
   or mixtures thereof. In addition, the honey can be pasteurized honey and/or filtered honey. The sugars optionally present in the mixture can be, in accordance with the definition above, sucrose, glucose, fructose and can be, for example, cane sugar or beet sugar or mixtures thereof.
   The inulin optionally present can be inulin derived from chicory, but also from burdock, dandelion, elecampane.
   The mixture according to the invention thus contains classes of functional components originating from medicinal plants and natural substances selected on the basis of the mechanical-physical effects that they are able to implement locally and that are able to intervene in the physiopathogenetic mechanisms triggering the cough and allowing it to persist in a self-maintained manner.
   The table below shows concisely each functional component and the physical-mechanical effect attributed by the inventors to such component.
   In the following paragraphs, the individual functional components and the scientific literature and the laboratory tests substantiating the activities attributed thereto will be examined.

| Components | Mechanical-physical effect |
|---|---|
| Resins | Barrier effect |
| Saponins | Mechanical stimulus of expectoration, reduction of |
| | surface tension of the mucus, direct fluidification of |
| | the secretions |
| Polysaccharides | Anti-adherent protective barrier for various pathogens, |
| | addition of water to the secretions |
| Sugars and honey | Adhesivity (viscosity-consistency), increase of |
| | salivation and of the amount of water in the mucous |
| | secretions |
| antioxidants | Protection against direct damage from free radicals |
| e.g.:polyphenols, | |
| flavonoids, | |
| vitamins | |

The effects of the resins are particularly pronounced for topical use, both directly as barrier effect and because they allow the other components to remain adhered for longer in contact with the irritated epithelium, preventing them from being washed away quickly by saliva or other liquids, allowing them to be effective for longer and to implement their mechanical-physical effect of protection of the mucosa against external agents to the best possible extent with relative indirect reduction of the inflammatory state. The saponins from a chemicophysical point of view are characterised by their particular property of acting in surfaces that, for the destination of use in the primary respiratory tracts, lead to an expectorant and antitussive effect, given the hydration of the mucosa and the assistance that they give to the expulsion thereof, thus resulting in having an effect of the mucoregulatory type.

In fact, due to their chemical characteristics, saponins are able to alter the permeability of the cell membranes: when they come into contact with the mucosa, they implement a threefold action:

1. stimulation of the expectoration reflex by irritation of the mucosa,

2. fluidification of the secretions by stimulation of the production of water;

3. reduction of the surface tension of the secretions, thus facilitating the detachment thereof from the mucosa.

The composition of the invention can be produced in any pharmaceutical form for oral use known in the field, such as in the form of a capsule, tablet, granules, powder, syrup, elixir, hard gelatine, soft gelatine, suspension, emulsion, solution.
In addition to the above-described active ingredients, the composition of the invention can comprise excipients of various type, preferably in pharmaceutically acceptable form.
Such excipients can vary in accordance with the embodiment of the base composition on the basis of the standard pharmacological knowledge of a person skilled in the art.
The excipients that can be present in the composition can be sweeteners, humectant agents, carriers, binding agents, diluting agents, disintegrating agents, lubricants, gliding agents, preservative agents, aggregants, flavourings, anti-adherent agents. Said excipients are preferably in pharmacologically acceptable form.
For oral administration, the composition can be produced in the form of a single daily dose or fractions of a single daily dose (for example 2, 3, 4, 5, 6 or more capsules, tablets, mono-dose lozenges of granulate or powder, syrup or fluid, or gelatins can be assumed during the day in accordance with the doctor's recommendation at the time) and may contain conventional excipients including, for example, binding agents, such as gum (tragacanth, Arabic gum), animal gelatine and polyvinylpyrrolidone; diluting agents such as sugar, polyalcohols (sorbitol, mannitol, xylitol), maltodextrins, inorganic salts (dicalcium phosphate, calcium carbonate); disintegrating agents such as rice starch, corn starch and potato starch; lubricants such as magnesium stearate, polyethylene glycol at various molecular weight; gliding agents such as colloidal silica; anti-adherent agents such as talc; humectants such as sodium lauryl sulphate.
By way of example, the composition can thus comprise one or more excipients such as (but not limited to) any combination of one or more of: preservative agents, when present, selected from one or more of: benzoates, parabens, sorbates; aggregating agents, when present, selected from one or more of: gums, natural and synthetic polymers, sugars; flavouring agents, when present, selected from one or more of: natural lemon flavour, sweet orange natural flavour, eucalyptus natural flavour, myrtle natural flavour, peach natural flavour; humectant agents, when present, selected from one or more of: sodium lauryl sulphate, sorbitan esters; sweeteners, when present, selected from one or more of: cane sugar, beet sugar, fructose; carrier agents, when present, selected from one or more of: water, demineralized water, deionized water; binding agents, when present, selected from one or more of: natural gums, for example tragacanth, Arabic gum, xanthan gum, animal gelatine, polyvinylpyrrolidone; diluting agents, when present, selected from one or more of: sugar, polyalcohols (sorbitol, mannitol, xylitol), maltodextrin, inorganic salts (dicalcium phosphate, calcium carbonate); disintegrating agents, when present, selected from one or more of: rice starch, corn starch, potato starch; lubricant agents, when present, selected from one or more of: magnesium stearate, polyethylene glycols; gliding agents, such as colloidal silica; anti-adherent agents, when present, such as talc.

When the composition is to be produced in the form of tablets, these can be coated in accordance with methods that are well known in normal pharmaceutical practice. The composition can also be produced in liquid form or semi-liquid form, suspension, emulsion, solution for oral administration, and optionally may contain natural aromatizing agents that confer a pleasant flavour to said composition.

The composition in the form of a powder or granulate for example can be pre-dosed in suitable containers and ready for use or for ingestion as such, or can be intended for resuspension in a suitable liquid, such as water or tea or the like. In this case too, the composition can contain natural aromatizing agents that confer a pleasant flavour to said composition.

In one embodiment the composition as described, in any of the above-mentioned embodiments, can be in the form of a pharmaceutical composition, or can be inserted into a special purpose food or into a medical device.

The composition according to the present description can be produced in the form of a pharmaceutical composition or in the form of a medical device according to any one of the classes described in EEC Medial Device Directive 93/42 (which also includes substances and not just “devices” in the sense of “objects”).

In the case of a syrup, in addition to all above-described active components, one or more of the agents described previously can also be used (preservative agents, aggregants, carrier agents, flavouring agents, etc.).

Formulations for adults or for children can also be produced.

In a particular embodiment of the present invention, the composition thus comprises a mixture of active ingredients formed from the following components:

adults and/or children

Antioxidants (polyphenols, flavonoids, vitamins) of
plant and/or natural origin
polysaccharides of plant origin
resins of plant and/or natural origin
saponins of plant origin
honey
optionally also sugars of plant origin and/or inulin

In one particular embodiment the flavours can be lemon flavour, sweet orange, myrtle, and lemon in formulations for children and eucalyptus flavour, star anise flavour and lemon flavour.
Some examples of compositions according to the invention are presented below.

### Composition Example 1

Composition comprising a mixture of active ingredients consisting of:
polysaccharides of plant origin between 0.1 and 0.25% by weight of said composition;
antioxidants of plant origin between 0.009 and 0.05% by weight of said composition;
resins of plant origin between 0.01 and 0.05% by weight of said composition;
saponins of plant origin between 0.0003 and 0.001% by weight of said composition;
honey between 20 and 35% by weight of said composition
further glucose, sucrose, fructose between 45 and 55% by weight of said composition
and one or more pharmaceutically acceptable excipients, humectants, carriers, binders, diluting agents, disintegrating agents, lubricants, gliding agents, preservative agents, aggregants, flavourings and/or anti-adherents as described above.
In this composition example, the flavourings can be essential oil of myrtle, of sweet orange, of lemon or mixtures thereof, for example.
The sugars added in addition to honey can be sucrose, fructose, glucose or mixtures thereof, and the sucrose can be present as cane sugar, beet sugar or mixtures thereof.
In one particular embodiment, this composition is produced in liquid form, for example as a syrup, and the excipients can comprise or consist of water, which is preferably deionised or demineralised.
In one embodiment of the invention, the antioxidants (polyphenols, in particular phenols, tannins, flavonoids or a mixture thereof, or also vitamins alone or combined with polyphenols) present in the mixture are derived from one or more of the following plants: Helychrisum italicum, Thymus vulgaris, Thymus serpyllum, Sambucus Nigra, Marrubium vulgare, Tilia cordata, Tilia platophyillos, Verbascum densiflorum, Glycyrrhiza glabra, Salvia officinalis, Echinacea purpurea, Plantago lanceolata, Grindelia robusta and/or from propolis.
In another embodiment of the invention, the polysaccharides are derived from one or more of the following plants: Plantago lanceolata, Aloe vera, Althaea officinalis, Malva syslvestris, Cetraria islandica, Verbascum densiflorum, Glycyrrhiza glabra, Tilia cordata, Tilia platophyillos, Echinacea pallida, Echinacea purpurea, Echinacea angustufolia, Helychrisum italicum, Salvia officinalis, Grindelia robusta.
In a further embodiment of the invention, the resins of the above-described mixture are derived from one or more of: Grindelia robusta, Commiphota myrrha, Boswelia serrata, Pinus mugo, Pinus sylvestris, propolis.
In yet a further embodiment, the saponins are derived from one or more of: Grindelia robusta, Verbascum densiflorum, Glycyrrhiza glabra, Marrubium vulgare, Poligala senega.
Lastly, the honey present in the mixture can be bee honey and/or honeydew honey. In a preferred embodiment, antioxidants, polysaccharides, resins and saponins are derived from the above-described sources.
For example, the antioxidants (polyphenols, in particular phenols, tannins, flavonoids or a mixture thereof or also vitamins alone or combined with polyphenols) can be derived from one or more of Helychrisum italicum, Grindelia robusta, Glycyrrhiza glabra, Plantago lanceolata, Thymus vulgaris, Sambucus Nigra; the polysaccharides can be derived from one or more of Helychrisum italicum, Grindelia robusta, Glycyrrhiza glabra, Plantago lanceolata, Echinacea pallida, Echinacea purpurea, Echinacea angustufolia, Thymus vulgaris; the resins can be derived from one or more of Grindelia robusta, Commiphota myrrha, Boswelia serrata and the saponins can be derived from one or more of: Grindelia robusta, Verbascum densiflorum, Glycyrrhiza glabra.
In accordance with another embodiment, the antioxidants (polyphenols, in particular phenols, tannins, flavonoids or a mixture thereof, or also vitamins alone or combined with polyphenols) can be derived from one or more (or also all) of Helychrisum italicum, Grindelia robusta, Glycyrrhiza glabra, Plantago lanceolata; the polysaccharides can be derived from one or more (or also all) of Helychrisum italicum, Grindelia robusta, Plantago lanceolata; the resins can be derived from Grindelia robusta, and the saponins can be derived from one or both of: Grindelia robusta and/or Glycyrrhiza glabra.

### Composition Example 2

Composition comprising a mixture of active ingredients consisting of:
polysaccharides of plant origin between 0.2 and 0.7% by weight of said composition;
antioxidants of plant origin between 0.02 and 0.1% by weight of said composition;
resins of plant origin between 0.04 and 0.1% by weight of said composition;
saponins of plant origin between 0.0009 and 0.002% by weight of said composition;
honey between 75 and 85% by weight of said composition
and one or more pharmaceutically acceptable excipients, humectants, carriers, binders, diluting agents, disintegrating agents, lubricants, gliding agents, preservative agents, aggregants, flavourings and/or anti-adherents as described above.
In this composition example, the flavourings can comprise or consist of essential oil of lemon, essential oil of anise, essential oil of eucalyptus or mixtures thereof, for example.
The sugars in this formulation are represented by only sugars present in the honey, and no further sugars are added.
In one particular embodiment, this composition is produced in liquid form, for example as a syrup, and the excipients can comprise or consist of water, which is preferably deionised or demineralised.
The classes of substances can be derived from plants and/or natural substances in each of the embodiments described above for example 1.

### Composition Example 3

Composition comprising a mixture of active ingredients consisting of:
polysaccharides of plant origin between 1.6 and 3% by weight of said composition;
antioxidants of plant origin between 0.1 and 0.8% by weight of said composition;
resins of plant origin between 0.1 and 0.8% by weight of said composition;
saponins of plant origin between 0.006 and 0.02% by weight of said composition;
honey between 3 and 5% by weight of said composition
further glucose, sucrose, fructose between 75 and 85% by weight of said composition
inulin between 1.5 and 2.5% by weight of said composition
and one or more pharmaceutically acceptable excipients, humectants, carriers, binders, diluting agents, disintegrating agents, lubricants, gliding agents, preservative agents, aggregants, flavourings and/or anti-adherents as described above.
In this example the flavourings can comprise or consist of flavours of eucalyptus, balsamic mint, essential oils thereof or mixtures thereof.
The sugars added in addition to those present in the honey can be sucrose, fructose, glucose or mixtures thereof, and the sucrose can be present as cane sugar, beet sugar or mixtures thereof.
The inulin can be extracted from chicory, but also burdock, dandelion, elecampane or mixtures thereof.
In one particular embodiment, this composition is produced in solid form, for example as a granulate, and the excipients may comprise gum, such as natural gums including tragacanth, Arabic gum, xanthan gum or mixtures thereof.
The classes of substances can be derived from plants and/or natural substances in each of the embodiments described above for example 1.

### Composition Example 4

Composition comprising a mixture of active ingredients consisting of:
polysaccharides of plant origin between 1.2 and 2.5% by weight of said composition;
antioxidants of plant origin between 0.1 and 0.6% by weight of said composition;
resins of plant origin between 0.3 and 1% by weight of said composition;
saponins of plant origin between 0.007 and 0.02% by weight of said composition;
honey between 5 and 8% by weight of said composition, and
further glucose, sucrose, fructose between 70 and 80% by weight of said composition and
inulin between 3.5 and 4.5% by weight of said composition (the percentage in the formula is 4%)
and one or more pharmaceutically acceptable excipients, humectants, carriers, binders, diluting agents, disintegrating agents, lubricants, gliding agents, preservative agents, aggregants, flavourings and/or anti-adherents as described above.
The further sugars (added in addition to honey) can be sucrose, fructose, glucose or mixtures thereof, and the sucrose can be present as cane sugar, beet sugar or mixtures thereof.
The inulin can be extracted from chicory, but also from burdock, dandelion, elecampane or mixtures thereof.
In this composition example the flavourings can comprise or consist of flavours of eucalyptus, balsamic mint, essential oils thereof or mixtures thereof.
In one particular embodiment, this composition is produced in solid form, for example as pills or tablets, and the excipients may comprise gum, such as natural gums including tragacanth, Arabic gum, xanthan gum or mixtures thereof.
The classes of substances can be derived from plants and/or natural substances in each of the embodiments described above for example 1.
In all of the above examples, the excipients are added as required to reach 100% weight.
A person skilled in the art, on the basis of the teaching provided in the present description, will be able to produce other compositions that fall within the scope of the present invention without particular difficulty or use of inventive skill.
The invention also relates to a process for preparing the composition in any embodiment described here in which extracts of ribwort plantain, grindelia and extracts of helichrysum are mixed together and with honey and one or more pharmaceutically acceptable excipients as defined above.
Lastly, the invention relates to a therapeutic method for the treatment of persistent cough as defined above, comprising the administration of therapeutically effective doses of the composition of the invention to patients.
The daily doses can be between 5 and 10 ml of composition in liquid form (example composition 1 or 2) once, twice, three times or four times per day, or even 2, 3 or 4 pills per day (each pill may weight between 1 and 2 grams, for example 1.5 grams, or 1, 2, or 3 pouches of granules per day (each pouch may weight between 1.5 and 3.5 grams, for example 2 grams).

Pre-clinical tests and clinical studies reported below have confirmed the efficacy of the composition of the invention in the treatment of persistent cough.

The efficacy of the composition and the possible mechanism of action thereof were examined by means of pre-clinical tests and clinical studies: tests for mucoadhesion, barrier/protection effect, test of protection against standardised tussigenic stimuli.

The adhesive and “barrier effect” properties of the product were investigated in order to confirm the hypothesis that mechanical-physical action of protection of the mucosa against irritative stimuli of external agents or internal irritants reduces the production of inflammation mediators and is effective in the treatment of persistent cough.

In order to validate the theory of the inventors, in accordance with which an effect of mechanical protection could be effective in the treatment of persistent cough, the ability of the composition to adhere temporarily and to implement a barrier effect was checked.

The resistance of the mucoadhesive layer formed by interaction between the components of the composition and the mucous cells was then assessed. The results obtained demonstrated an interesting ability of the composition to remain bioadhesive to the mucosa. When the composition was subjected to washing with artificial saliva, the process of bioadhesion persisted for one hour. From the tests performed, the composition thus shows that it possesses good mucoadhesive power both in pure form (not diluted) and when diluted 1:2 and 1:5.

It was elected to use the composition also in diluted form to simulate the process of washing to which said composition can be subjected by saliva and during swallowing. The composition diluted 1:2 remains on the experimental oral mucous membrane for a length of time sufficient to guarantee protection.

Even at extreme dilution (1:5), practically impossible to produce by means of normal salivation, a noteworthy percentage of adhesion ability was observed. On the basis of the drop in mucoadhesion observed among the various recordings, it is fair to state that the adhesion of the product to the mucosa lasts for approximately 60 minutes per administered dose.

The results obtained from the mucoadhesion test make it possible to confirm that the composition of the invention is able to limit the exposure of the mucosa to irritant agents and, by means of its filmogenic activity, also limits the dehydration of the mucosa, implementing a local anti-inflammatory effect of the non-pharmacological, non-immunological and non-metabolic type.

Again, in order to check the mechanism of action of the composition, the protective filmogenic capability thereof with respect to a conventional irritant agent, constituted in this case by lipopolysaccharide (LPS), a significant component of the outer membrane of gram-negative bacteria, was tested in accordance with a conventional model of inflammatory induction. The object of the test was to identify the effective capability of the composition to limit contact between the mucosa and external irritant agents. To assess the protective action of the composition relative to the antigen LPS, the production of interleukin 6 and interleukin 8 (IL-6 and IL-8) was measured, these being formed as a result of the contact between a layer of cells (fibroblasts) and LPS. Il-6 is more specific for this type of attack, and therefore the inhibition thereof represents a direct measure of the barrier effect. The results of the study demonstrated a significant reduction of the production of IL-6 and IL-8 caused by the fact that the composition is arranged between the cells and the stimulating agent, thus reducing production of the two mediators of inflammation and thus exerting an indirect anti-inflammatory action (by barrier effect).

The demonstration that the composition has protective action in relation to this antigen makes it possible, given the dimensions of lipopolysaccharide (LPS), to confirm that the product can exert a barrier action in relation to other antigens and irritant agents as well, such as powder, smog and pollen.

In order to test in vivo the protective efficacy against irritative stimuli of the composition, an experimental test of cough induction using an irritant was carried out at the University of Milan.

The objective of the study, which was performed on mice, was to verify the protective power of the composition on mucosa subjected to irritant stimuli known for their ability to stimulate coughing, such as capsaicin, observing at the same time the susceptibility of the mucosa to various irritant stimuli. The action of the composition was compared with that of two common substances with central sedative effect (codeine and dextromethorphan), honey and a placebo.

The analysis of the results of the tests confirmed the protective action of the composition and the ability thereof to reduce the fundamental susceptibility of the mucosa to acute irritative stimuli. The protection and the reduction of the sensitivity to the tussive stimulus manifested itself both immediately after administration, this being “acute” treatment, and during continued treatment, “continuous”.

The antitussive action of the composition was almost as effective and significant as that of the two sedative drugs both in the “acute” treatment and in the “continuous” treatment and was greater than that of honey and the placebo in a statistically significant manner (p<0.001) in both treatments. The effect does not appear to be dose-dependent. The best performance of the composition compared with honey, whether pure or one of the main components thereof, can be explained in the synergism of action between the various components.

A double-blind randomised multi-centre clinical study was performed against placebo to verify the efficacy of the composition of the invention on acute persistent cough (from 1 to 3 weeks) and was approved by the Ethics Committee of Udine University Hospital and was conducted in accordance with the following directives:

1. Declaration of Helsinki concerning medical research in humans (‘Recommendations Guiding Physicians in Biomedical Research Involving Human Patients’, Helsinki 1964, amended Tokyo 1975, Venice 1983, Hong Kong 1989, Somerset West, 1996, Edinburgh, 2000, Washington, 2002, Tokyo, 2004, Seul, 2008).

2. UNI EN ISO 14155-1:2009

3. UNI EN ISO 14155-2:2009

4. UNI EN ISO 14155:2012.

The study enlisted 102 children (aged between 3 and 6 years and for whom parental consent was obtained) who presented a cough for at least one week, but less than three weeks. Of these children, 51 received the product and 51 received the placebo (5 ml for 4 times per day). The day and night cough score survey in accordance with the appropriate Chung questionnaire was carried out both by means of specialist visit by the doctor and by means of the compilation of a diary by the parents at times T0, T4 and T8.

With regard to the inter-group analyses of the children treated with the composition of the invention compared with the children treated with placebo, a statistically significant difference was observed in the night cough score at time t4 (p=0.03). The intra-group analysis in the ITT population of the group of children treated with the composition of the invention revealed a statistically significant difference in the day cough score (t0 vs t4: p=0.04, t4 vs t8: p=0.01 and t0 vs t8: p=0.001), confirming how the product being studied appears to be effective at times t4 and t8, thus at days 4 and 8 from the start of treatment. This result confirms the efficacy of the composition, considering that children suffering from persistent cough for at least 7 days were treated, which, as is known, is generally more difficult to resolve. With regard to the intra-group analyses of the children treated with the composition of the invention, a statistically significant difference in the night cough score was observed at time t4 (t0 vs t4: =0.003) and in the comparison of times t4 vs t8 (p=0.05) and t0 vs t8 (p=0.005). Given the disturbance that night cough causes to a child and also given the difficulty generally encountered in the treatment of this symptom, the result obtained with the composition of the invention after just 4 days of treatment is decidedly important. Considering instead the intra-group analysis in the ITT population for the group of children treated with the placebo, it can be noted that the placebo did not demonstrate any statistically significant efficacy at the endpoints: t0 vs t4 (day; p-value=0.3) and t0 vs t4 (night; p-value=0.2).

In the group treated with the placebo, a statistically significant difference in the score, both of day cough and night cough, was observed only after 8 days of treatment, but this can be considered the effect of the process of natural resolution of the cough.
The results therefore demonstrate the superior efficacy of the composition of the invention compared with the placebo: in the group of children treated with the composition of the invention, the cough was reduced from the start of the treatment and the children treated had significantly improved from T0 to T4, whereas the group of children treated with placebo demonstrated significant improvements only after 8 days (due to a probable natural resolution of the cough).

The decrease of the cough at t4 in the group treated with the composition of the invention confirms that the protective role of the product promotes the resolution of the symptom in relatively short periods of time. This effect is particularly evident and interesting on night cough because it allows the child the rest better.

It is important to stress in addition that the children in the study at the moment of enrolment had already been suffering from a cough for a week. These then form that 50% of children who, according to Hay and Wilson (reference already cited), have not improved spontaneously over the course of the first two days of illness and therefore are developing a persistent cough. In light of these positive results and the comparison with the information obtained with the placebo, the composition can be used specifically for the treatment of acute persistent cough and is effective already from the first days of treatment.

Comprehensive reading of the results of the study makes it possible to confirm significant efficacy of the composition of the invention in the soothing of acute cough and persistent cough and in particular night cough from the 4th day of treatment (day of the first clinical check following inclusion in the study). With regard to that mentioned in relation to the disturbance and suffering that a cough can cause in a child and in the child's family, in particular a persistent night cough, it is also fundamental to highlight that the composition does not have any problems with regard to safety and can help to improve the condition of health and the quality of life of the child.

In conclusion, it is possible to confirm that the composition of the invention constitutes an innovative method for tackling persistent cough, for example accompanying URTI, and that it has been proven to be effective even on a population of children suffering from persistent cough for more than a week.

The efficacy of the actions is demonstrated by pre-clinical studies in vitro and in vivo on animals and clinical studies on children. On the whole, these studies have confirmed the significant efficacy of the composition in soothing intense irritative stimuli (aerosol of capsaicin) both in acute treatment (1 single dose 30 minutes before applying the irritative stimulus) and in continuous treatment (2 doses per day for 3 consecutive days prior to application of the irritative stimulus). It was also observed that the ability of the composition to protect against irritative stimuli lasts over time, and in fact both in the “acute” treatment and in the “continuous” treatment it was observed that the composition reduced coughing fits in a statistically significant manner even at a distance of 30 minutes from the application of the irritative stimulus. The effects of the composition were always greater than those of honey, reinforcing the hypothesis that the actual efficacy of the product lies in the synergy of action of the mixture of natural molecular complexes contained in the composition.

The pre-clinical studies have also confirmed the properties of mucoadhesion and the barrier effect of the composition that were considered to be fundamental by the inventors in order to compare the etiopathogenetic mechanisms of persistent cough.

The clinical study in children has demonstrated that the composition is more effective than the placebo in a statistically significant manner (p=0.03) in soothing night cough in children on the 4th day of treatment. Night cough is the symptom that is the most bothersome and the most difficult to endure for children and for parents and causes significant disturbance of the quality of life of both. The composition also has superior efficacy compared to the placebo when it comes to soothing day cough in children that persists beyond 7 days, already from the 4th day of administration.

With regard to the problems of safety, it is useful to remember that the composition does not irritate the oral mucous membrane, does not cause sensitisation, is not cytotoxic and is not toxic for oral administration at concentrations >2000 mg/kg.

None of the classes of substances contained in the product (resins, polysaccharides, sugars of honey, saponins and antioxidant substances as defined above) has effects on the respiratory system that could in some way arouse concern. The mechanism of action of the mechanical-physical type and the effect of facilitation of expectoration without alteration of the structure of the mucosa does not in any way cause fear of adverse effects of the type observed with the drugs.

The following examples present possible embodiments of the invention which are deemed to be the best embodiments and are to be considered to provide a person skilled in the art with a starting point for the purpose of repeating the invention, but are in no way limiting. A person skilled in the art, on the basis of the teaching provided here, will be able to produce other compositions envisaged by the present invention without the need to carry out particular research or exercise inventive skill.

### EXPERIMENTAL EXAMPLES

The examples presented below were carried out with composition examples 1-4 described above.
The test on children was carried out with composition example 1 insofar as this is the most suitable composition for children on account of taste.

### 1. Pre-Clinical Tests on Cough

To test the efficacy in vivo of the product, a test was carried out by means of induction of cough using an irritant.
A study was carried out on mice to verify the protective power of the mixture of the invention in relation to irritant tussive stimuli, such as capsaicin, and therefore to verify the susceptibility of the mucosa to irritant stimuli.
In accordance with a test described by Kamei and performed in 2003, female mice (CD-1) were divided randomly into seven groups as specified below; each group was formed by 5 mice.
The verification of the antitussive power of the samples was performed both acutely, administering the tested product 30 minutes prior to the tussive stimulus, and “continuously”, administering the tested product twice per day for three days and inducing the tussive stimulus 4 hours following the last administration.
Each of the 7 groups of mice was first subjected to the acute treatment and then to the continuous treatment:
The acute administration of the tested product (that is to say 30 minutes prior to the tussive stimulus) was performed for the purpose of confirming the protective efficacy of said product in relation to an irritant stimulus.
The continuous administration of the product, with application of the tussive stimulus 4 hours following the last administration (beyond the residence time of said product at the mucosa), makes it possible to verify the efficacy of the tested product in protecting a mucosa normally exposed to irritative or dehydrating ambient stimuli, reducing the basic susceptibility thereof to acute irritative stimuli.
   The results allow a comparison of efficacy between the products tested, independently of their mechanism of action. It should be remembered in fact that codeine and dextromethorphan exhibit action at central level, whereas honey and the components of the mixture of the invention do not have such action.
   The tested products were applied by tube to the entrance of the pharynx so as to allow the samples exerting protective action to adhere to the epithelium covering the walls of the pharynx.

### Acute Treatment:

administration of the tested product 30 minutes prior to the tussive stimulus and counting of the coughing fits immediately and 30 minutes after the end of the stimulus.

### Groups (5 Mice/Group). Treatment Per Mouse.

1. without protective treatment

2. 8 micrograms of dihydrocodeine rhodanide

3. composition of the invention in 50 microlitres of liquid formulation

4. composition of the invention in 100 microlitres liquid formulation

5. ABO/BRO (product based on dextromethorphan) 50 microlitres

6. honey 100 microlitres

7. placebo 100 microlitres

The test was carried out in two periods.

### Continuous Treatment:

administration of the product 2 times per day for a total of 100 microlitres/day and administration of the tussive stimulus 4 hours after the last administration of product, on day 3.

### Groups (5 Mice/Group). Treatment Per Mouse

1. without protective treatment

2. 8 micrograms of dihydrocodeine rhodanide

3. mixture of the invention in liquid formulation 25 microlitres twice per day

4. mixture of the invention in liquid formulation 50 microlitres twice per day

5. ABO/BRO (product based on dextromethorphan) 24 microlitres twice per day

6. honey 50 microlitres twice per day

7. placebo 50 microlitres twice per day

### Induction of the Cough

The animals were treated with capsaicin (Sigma-Aldrich) 45 μM (30 μg/ml in 10% alcohol in saline solution), administered for 3 minutes by means of aerosol. The coughing fits were counted using a neonatal stethoscope (Quirumed S. L.)
To allow the contact of the products with the oral mucous membrane, the products were administered by means of a tube at the pharynx.
The samples were administered in pure form, whereas honey, which is too dense, was diluted 1:3 in order to reduce the density thereof.

### Results

The results, which confirm the protective nature of the composition of the invention, are presented below. The protection and the reduction of the sensitivity to the tussive stimulus manifest themselves both immediately following administration and during continuous treatment over three days.

### Acute Treatment

After one minute from the application by aerosol, the effect of the capsaicin was first observed, which is a cough inducer, then the efficacy of codeine and of dextromethorphan in eliminating the cough by means of the known mechanism of central type. The antitussive effect of the mixture of the invention was also noted, which slowly increases with the dose (50 microlitres of the mixture of the invention reducing the cough by 35% whereas 100 microlitres reduce it by 46%), likely caused by a non-saturation of the sites of adhesion of the lower dose, and therefore a greater protection provided by the higher dose. Both results are statistically significant to the same extent. It is important to note that the activity of honey, known to protect the throat, was compared and demonstrated protective action in relation to the tussive stimulus, although to a lower extent compared with the mixture of the invention. This result shows that the beneficial action of the mixture of the invention is linked to the protective synergy of the components present and not only to the action of the honey.

The placebo did not demonstrate any effect.
The results are presented in FIGS. 2 and 3.
After 30 minutes from the acute administration of capsaicin, it was observed that: the cough decreases even in the group not treated with any sample; in addition, it is noted that the honey loses efficacy, that the mixture of the invention maintains its efficacy and that a greater efficacy of the administration at higher dose is also highlighted, as if a greater quantity of product were able to provide an improved protective effect. The agents with central action maintain their efficacy in a substantially unchanged manner.
For the interpretation of the results compared to the measurement after one minute (reduction of the number of coughing fits), the reduction of the irritative stimuli on the throat caused by the increase of the secretions of the pharynx and of the close tissues was taken into consideration. ABO/BRO is a product based on dextromethorphan.
The effect of the mixture of the invention lasts over time, whereas the effect of honey is exhausted within 30 minutes, demonstrating the synergy between honey and the other components of the product.
The results are shown in FIGS. 4 and 5.
In the continuous treatment it was first noted that the effect on coughing is not modified compared to the acute treatment: on average, the mice that had not received treatment had the same response as three days prior.
The protective effect of the mixture of the invention is demonstrated by the fact that, despite whether the administration was performed 4 hours from the tussive stimulus with capsaicin, the results obtained in the group treated with the same mixture are comparable with those obtained in the acute treatment.
Codeine and ABO/BRO 50 lose their efficacy slightly, probably due to the minor presence at the site of action on account of pharmacokinetics. Honey and the placebo maintain the performances witnessed with acute treatment.
The results are presented in FIGS. 6 and 7.
FIG. 8 shows the response in the continuous treatment after 30 minutes from capsaicin. As can be seen in the figure, the experiment has showed a trend similar to the acute treatment: the cough reduces physiologically, honey loses efficacy, the products with central action substantially maintain the efficacy witnessed 30 minutes prior, the mixture of the invention has improved performance, probably due to the fundamental protection of the mucosa, which makes it possible to positively modulate the sensitivity thereof to irritant stimuli. In this case, a greater efficacy of the higher doses is witnessed, probably due to a greater presence of the product over time on the mucosa, the product thus being able to produce a greater protective effect.

### 2. Mucoadhesion Test

To verify the mechanism of action forming the basis of the efficacy of the mixture of the invention, tests were carried out in vitro on the adhesive properties and barrier of the product, so as to verify the benefit of that reported in the literature regarding the importance of protecting (in a clinic) the throat against external or internal irritant agents (the internal irritant agents include, for example, post-nasal drip, which causes cough of the persistent type).
The results of the tests are summarized here and are described in greater detail further below.
The product demonstrates good mucoadhesive power both in undiluted form and at a dilution of 1:2 and dilution of 1:5.
In addition, the product demonstrates a good duration of the mucoadhesion.
   The tests were performed in accordance with a specific experimental model based on the specific mechanisms of the mucoadhesion described below in detail;
   The mucoadhesive effect of a product, which contributes to the formation of the protective film on mucosa, can be assessed by means of suitable models in vitro. The model used demonstrates that the mucoadhesion of products intended for the treatment of mucosa can be determined by means of the assessment of the percentage of inhibition of the lectin-glycoprotein bond. Initially, the mucous cells in the mouth are treated with biotinylated lectin (Con-A), a protein contained in some leguminosae (Canavalia ensiformis) that has a high affinity for the 15-glucoside and mannoside residues present in the membrane glycoproteins. In this way, the sites of the glycoproteins of the mucous membranes are all occupied by biotinylated lectin (treated with biotin, that is to say vitamin H). The cells, treated with biotinylated lectin, are mixed with streptavidin peroxidase such that, thanks to the high affinity between biotin and streptavidin, the complex protein/glucose/lectin/biotin/streptavidin peroxidase is formed. At this point, the cells are washed and the protein/glucose/lectin/biotin/streptavidin peroxidase complex is quantified, assessing the activity of the peroxidase by means of the reaction of oxidation of the ortho-phenylenediamine (colorimetric assessment). In fact, the protein/glucose/lectin/biotin/streptavidin peroxidase complex will catalyse the oxidation reaction:

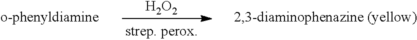

The intensity of the yellow/orange colouration of the solution (measured using a spectrophotometer at λ=450 nm) is proportional to the quantity of glycoprotein/lectin bonds and therefore to the quantity of available sites (glycoproteins) for mucoadhesion. The absorbency value thus determined constitutes the “control”.
When determining the mucoadhesion of the product, the cells are treated preliminarily with this product (incubation at 30° C. for 15 minutes before the treatment with lectin). If the product under examination contains mucoadhesive substances, these bond to the glucoside and mannoside sites present in the glycoproteins of the membrane. In the next phase, constituted by adding the sequence of biotinylated lectin, streptavidin peroxidase and ortho-phenylenediamine, it is possible to obtain a less intense colouration compared to the control, since some of the glucoside sites available for bonding with the Con-A were already occupied by the mucoadhesive substances present in the product to be tested. In fact, the initial bonding between the mucoadhesive substances contained in the product to be tested and the glucoside sites partly compromises the subsequent conjugation of the Con-A with the streptavidin peroxidase complex and the subsequent development of colour after addition of oxygenated water.

The decrease in the absorbency value is proportional to the ability of the substances under examination to “mucoadhere” to the mucous cells. The mucoadhesive ability is expressed as a percentage of inhibition of glycoprotein/lectin bonding and represents the percentage of mucous sites occupied by the product in accordance with the following equation: percentage of mucoadhesion of the product=(1−abs sample/abs control)×100 Furthermore, in addition to the mucoadhesive ability, the resistance of the mucoadhesive layer to the action of the saliva solution with which it comes into contact is also assessed. To this end, in a second phase of the experiment, the resistance over time (0.5-2 h) of the mucoadhesion of the product following exposure to a continuous flow of artificial saliva solution was evaluated. To carry out this test, a system of Franz cells was used, these cells generally being used in the assessment of the process of percutaneous absorption of a substance or for the study of other processes of permeation through natural or artificial membranes.

In the experiment, the mouth cell cultures were deposited in the donor and were treated with the product as such, diluted 1:2 (dilution most likely closest to the actual conditions that could be verified in vivo) and diluted 1:5. The donor was then supplied with a continuous flow (2 ml/min) of artificial saliva solution by means of a peristaltic pump. At the base of the donor, in the zone of separation with the receptor, a membrane of cellulose acetate was arranged, able to allow the discharge of the saliva solution from the donor to the receptor, retaining in the donor the mucous cells. The saliva flow through the mucous cells treated with the product under examination was interrupted regularly after 0.5, 1 and 2 hours, and the cells of the donor were transferred to a suitable test tube for the assessment of the mucoadhesion.
In light of the results obtained, it is possible to confirm that the product, demonstrating possession of good and resistant mucoadhesion, appears able to provide a beneficial protective role in relation to the mucous cells of the oral tract.
For the purpose of mimicking the natural dilution of a syrup in the oral cavity, the mucoadhesion tests were performed on product diluted 1:2, and for greater completeness the mucoadhesion of the product diluted 1:5 was also tested. The percentage of mucoadhesion of the diluted product (1:2 and 1:5) in relation to human mouth cells is presented in FIG. 9. The resistance of the mucoadhesive layer obtained with the product diluted 1:2 at different times 0.5 h, 1 h and 2 h in relation to a simulated saliva solution (physiological solution of NaCl 0.9%) is shown in FIG. 10.
The objective of the dilution of the product was to minimise the “washing” encountered due to swallowing.
The residence times of the product diluted 1:2 on the experimental oral mucous membrane are also presented, from which it is possible to deduce a protection of approximately one hour implemented on the mucosa.
The data obtained therefore show (see FIGS. 9 and 10) that the mixture of the invention, which has a non-pharmacological, immunological or metabolic mechanism of action, limits the exposure of the mucosa to irritant agents and, thanks to the filmogenic activity thereof, also limits the dehydration of the mucosa, demonstrating a local anti-inflammatory effect of non-pharmacological type.
FIG. 9 shows the mucoadhesion of the mixture of the invention on oral mucous membrane at various dilutions, the measurement showing the percentage of adhesion of the mixture in liquid form (syrup) at the bonding sites of the oral mucous membrane depending on the dilution of the product.
FIG. 10 shows the resistance of the mucoadhesion to washing with simulated saliva.
The graph shows the percentage of adhesion of the mixture of the invention in liquid form (syrup) diluted 1:2 at the bonding sites of the oral mucosa: resistance to washing with artificial saliva 2 ml/min for a period up to 2 hours.

### 3. “Barrier Effect” Protection Test

Again in order to verify the mechanism of action of the product, the filmogenic protective capability of the product was tested compared to a conventional irritant agent, constituted by the membrane of lipopolysaccharide (LPS), which is a standard model of inflammatory induction. This test reveals the effective capability of the product to limit the contact between the mucosa and the irritant external agents. This irritant agent (which is infectious in this case) was taken from a portion of lipopolysaccharide of the cell membrane of Escherichia coli. The dimensions of the lipopolysaccharides (LPS) are such that the barrier can be considered useful for powder, smog, pollen, etc.
In synthesis, the results show
a decrease of the production of IL6 and
a decrease of the production of IL8 by contrast with contact between provocative agent and cells.
   More specifically, the production of IL6 and IL8 as a result of the contact between a layer of cells (fibroblasts) and LPS was measured. IL6 is more specific for this type of attack, and therefore the inhibition of IL6 is a direct measure of the barrier effect. The results, performed against negative, show that the presence of the mixture of the invention on a membrane arranged above the cells reduces the production of IL6 (produced by the cells following specific stimulation). This indicates that the mixture exerts a barrier effect that in part prevents the irritant agent from passing through the membrane.
   To a lesser extent, the production of IL8 is also inhibited, this being a cytokine that is always present in cells because it is produced as a constituent thereof. Precisely due to this pre-existence in the cells, an inhibition of IL8, even to a minor extent, shows that the product exerts a barrier effect in relation to LPS.
   FIG. 11 shows the protective effect of the mixture during formation of a barrier film measured in the experiment.
   The experiment has shown that the action of the sample produces an inhibition of the release of IL6 equal to 22.2% at the concentration of 2.5 μg/ml of LPS and of 13.4% of IL8.
   The difference of such diverse inhibitions is dependent on the different role of the interleukins in vivo, as mentioned above.
   The barrier effect of the product is thus confirmed. The certainty that such a reduction in the production of cytokines is caused by a barrier effect and not by direct anti-inflammatory action of the sample is provided by the internal control, where the order in which the cells are exposed to LPS and to the sample is reversed compared to the barrier experiment. In this control, the product is placed on the membrane in order to provide the barrier following stimulation with LPS. In such a condition there is no reduction of the interleukins, showing that the test highlights a barrier effect and not direct anti-inflammatory contact.
   It is therefore possible to confirm that the barrier efficacy of the sample is based on the absence of cell response due to the minor concentration of the allergen LPS following exposure.

### 3. Clinical Study on Children: Double-Blind Randomised Multi-Centre Study Versus Placebo to Assess the Efficacy and the Tolerability of the Composition of the Invention in Persistent Cough in Children

A double-blind randomised multi-centre study was carried out versus placebo in order to verify the efficacy of the mixture of the invention in persistent cough (from 1 to 3 weeks).
The study enlisted 102 children (aged between 3 and 6 years) who presented a cough for at least one week, but less than three weeks. Of these children, 51 received the product and 51 received the placebo (5 ml for 4 times per day). The day and night cough score survey in accordance with the appropriate Chung questionnaire was carried out both by means of specialist visit by the doctor and by means of the compilation of a diary by the parents at times T0, T4 and T8.

Inclusion Criteria

acute cough from 1 to 3 weeks

informed written consent from the parents or legal guardian

parents or legal guardians agreed not to administer any other products to the child for the treatment of cough in the period of study unless clinically necessary (in this case the patient was withdrawn from the study)

the parents or legal guardians had to follow the study protocol.

Exclusion Criteria

patients using immunostimulants or immunoregulators of biological origin in the month prior to the study

patients with congenital or acquired immunodeficiency

patients with chronic diseases

patients treated with antibiotics

patients treated with systemic corticosteroids

patients with potential viral diseases in the month prior to the study

patients who had taken part in clinical studies with experimental protocols in the month prior to the study

patients with suspected or diagnosed asthma

patients with bacterial pathologies (pneumonia, sepsis)

patients with positive medical history of allergy to plants belonging to those used to obtain the classes of compound of the mixture or to components thereof

inability of the patient or of the family to follow the protocol of the study

Statistical methods: changes between the three groups of treatment were measured by means of the Student's t-test for normally distributed variables and Wilcoxon rank sum test for those not normally distributed (the distribution of each parameter was assessed using the Kolmogorov-Smirnov test). The changes of the instrumental pre-post treatment parameters were assessed using the Student's t-test for samples or Wilcoxon Signed Rank test paired together depending on the cases.
The study carried out shows that the synergic action of the components according to the invention directly protects the pharyngeal mucosa from irritant stimuli, providing the mucosa with a protective shield that reduces the intensity of the cough and that does not present the systemic side effects associated with the administration of cough sedatives.
The composition also increases the aqueous component of the mucus, making it less viscous and easier to remove from the cilia of the respiratory cells.
The placebo syrup used contained demineralised water, xanthan gum, citric acid, cane sugar, potassium sorbate (E202), acesulfame K, lemon and orange flavour, vegetable carbon and beta carotene.
The composition used for this test corresponds to composition example 1.

### Clinical Scoring of Day Cough:

| | |
|---|---|
| 0 | Absent |
| 1 | For a brief period (approximately 2 minutes) |
| 2 | For 2 brief periods (approximately 10 minutes) |
| 3 | Frequent cough that does not interfere with normal activity |
| 4 | Frequent cough that interferes with normal activity |
| 5 | Annoying cough for the majority of the day |

### Clinical Scoring of Night Cough:

| | |
|---|---|
| 0 | Absent |
| 1 | Only on wakening/only before falling asleep |
| 2 | Awakening once/awakening early caused by the cough |
| 3 | Frequent awakening caused by the cough |
| 4 | Frequent cough for the majority of the night |
| 5 | Annoying cough |

102 patients were assessed: 51 with the composition of the invention general composition example 1, and 51 with the above-described placebo.

The study was carried out to the end by 44 patients with placebo and 47 with composition.
No patient left the study for safety reasons.
With regard to the inter-group analyses of the children treated with the composition of the invention, compared to the children treated with placebo, a statistically significant difference was observed in the night cough score at time t4 (p=0.03). The intra-group analysis in the ITT population revealed a statistically significant difference in the day cough score (t0 vs t4: p=0.04, t4 vs t8: p=0.01 and t0 vs t8: p=0.001), confirming that the product being studied appears to be effective at times t4 and t8, thus at days 4 and 8 from the start of treatment. This result, which is certainly of interest, confirms the efficacy of the product, considering that children suffering from persistent cough for at least 7 days were treated, which, as discussed above, is more difficult to resolve.
With regard to the intra-group analyses of the children treated with the mixture of the invention, a statistically significant difference in the night cough score was also observed at time t4 (t0 vs t4: =0.003) and in the comparison of times t4 vs t8 (p=0.05) and t0 vs t8 (p=0.005).
Given the disturbance that night cough causes to a child and also given the difficulty generally encountered in the treatment of this symptom, the result obtained with the mixture of the invention after just 4 days of treatment is very important.
Considering instead the intra-group analysis for placebo in the ITT population, it can be noted that the placebo did not demonstrate any statistically significant efficacy at the endpoints: t0 vs t4 (day; p-value=0.3) and t0 vs t4 (night; p-value=0.2).
In addition, the treatment with the mixture of the invention did not have any side effect, confirming the safety of the product, which has proven to be effective in soothing the cough from the start of the treatment. From the first check visit (T4 of the treatment), a statistically significant difference was observed for the night score between the children treated with the mixture of the invention and those treated with placebo.
The statistically significant difference of efficacy was demonstrated in the night and day scores in the treatment group compared to the initial visit (T0). The placebo group instead did not reach a significant improvement by day 4 compared to the initial visit (T0).
The decrease of persistent cough at t4 in the group treated with the mixture of the invention confirms the efficacy of the product, which promotes the resolution of the symptom in relatively short periods of time.
This effect is particularly evident and beneficial in the case of night cough because it allows the child to rest better.
As already indicated, the children in the study at the moment of enrolment had already been suffering from a cough for at least a week. In light of these positive results and the comparison with the information obtained with the placebo, the product forming the basis of the invention thus can be used specifically to provide relief to persistent cough and seems to be effective already from the first days of treatment.
It can be concluded that treatment with the composition according to the invention is safe and effective in reducing persistent cough, already from the start of the treatment itself, and is characterised by statistical significance of the night scores compared to the placebo in the first check visit on day 4 (tf) and of the day and night scores compared to the initial visit (t0). The placebo group does not reach significant levels of improvement at t4 compared to the initial visit. The decrease of the cough at t4 in the treated group shows the effect of the composition of the invention in the reduction of the tussive symptom, thus demonstrating an improvement in health. This effect is also more evident at night, allowing the patient to have better sleep quality since it prevents irritant stimuli.

### BIBLIOGRAPHY

Anderson-James S, Marchant J M, Acworth J P, Turner C, Chang A B Inhaled corticosteroids for subacute cough in children Cochrane Database Syst Rev. 2013 Feb. 28; 2:CD008888
Bolser D C. Cough suppressant and pharmacologic protussive therapy: ACCP evidence-based clinical practice guidelines. Chest. 2006 January; 129(1 Suppl):238S-249S.
Bruton I., Chabner B. A., Knollmann B. C. GOODMAN & GILMAN LE BASI FARMACOLOGICHE DELLA TERAPIA. Zanichelli, 2012, 12^{th} edition.
Carney I K, Gibson P G, Murree-Allen K, Saltos N, Olson L G, Hensley M J. A systematic evaluation of mechanisms in chronic cough. Am J Respir Crit Care Med. 1997 July; 156(1):211-6.
Chang A B. Cough: are children really different to adults? Cough. 2005 Sep. 20; 1:7.
Chung in Redington A E, Morice H. 2005. Acute and chronic cough. Taylor & Francis. Pag. 215-236.
Dicpinigaitis P V, Colice G L, Goolsby M J, Rogg G I, Spector S L, Winther B. (2009). Acute cough: a diagnostic and therapeutic challenge. Cough. 2009 Dec. 16; 5:11.
Hay A D, Wilson A, Fahey T, Peters T J. 2003. The duration of acute cough in pre-school children presenting to primary care: a prospective cohort study. Family Practice Vol. 20, No. 6.
Johnstone K J, Chang A B, Fong K M, Bowman R V, Yang I A. Inhaled corticosteroids for subacute and chronic cough in adults; Cochrane Database Syst Rev. 2013 Mar. 28; 3:CD009305. Review.
Kamei J, Morita K, Saitoh A, Nagase H. The antitussive effects of endomorphin-1 and endomorphin-2 in mice. Eur J Pharmacol. 2003 Apr. 25; 467(1-3):219-22
Lee P. C. L, Jawad M. S. M, Eccles R. 2000. Antitussive efficacy of dextromethorphan in cough associated with acute respiratory tract infection. J Pharm Pharmacol. 52:1137-1142.
Madison J M, Irwin R S. Pharmacotherapy of chronic cough in adults. Expert Opin Pharmacother 2003; 4:1039-1048.
Mu L, Sanders I. (2000). Sensory nerve supply of the human oro- and laryngopharynx: a preliminary study. Anat Rec. 2000 Apr. 1; 258(4):406-20.
Pratter M R. Cough and the common cold: ACCP evidence-based clinical practice guidelines. Chest. 2006 January; 129(1 Suppl):72S-74S.
Testagrossa O, Siclari E. 2005. Farmaci contro la tosse? Non sono efficaci. Quaderni acp; 12(4): 178-181.
Wheeler-Hegland K, Pitts T, Davenport P W. (2010) Cortical gating of oropharyngeal sensory stimuli. Front Physiol. 2010 Jan. 25; 1:167.
Yoder K E, Shaffer M L, La Tournous S J, Paul I M.). 2006. Child assessment of dextromethorphan, diphenhydramine, and placebo for nocturnal cough due to upper respiratory infection. Clin Pediatr (Phila). 45(7):633-40

1-17. (canceled)

18. A method of treating persistent cough comprising: administering, to a patient in need thereof, a composition comprising, as active ingredients, a mixture consisting of:
polysaccharides of plant origin between 0.06 and 5.5% by weight of said composition,
antioxidants of plant origin and/or natural origin between 0.005 and 1.2% by weight of said composition,
resins of vegetable origin and/or natural origin between 0.008 and 1.5% by weight of said composition,
saponins of plant origin between 0.0002 and 0.03% by weight of said composition,
honey between 3 and 85% by weight of said composition, and
glucose, sucrose, fructose, and/or inulin between 0 and 91% by weight of said composition.

19. The method according to claim 18, wherein said antioxidants are derived from one or more of: Helychrisum italicum, Thymus vulgaris, Thymus serpyllum, Sambucus Nigra, Marrubium vulgare, Tilia cordata, Tilia platophyillos, Verbascum densiflorum, Glycyrrhiza glabra, Salvia officinalis, Echinacea purpurea, Plantago lanceolata, Grindelia robusta, or propolis.

20. The method according to claim 18, wherein said polysaccharides are derived from one or more of: Plantago lanceolata, Aloe vera, Althaea officinalis, Malva syslvestris, Cetraria islandica, Verbascum densiflorum, Glycyrrhiza glabra, Tilia cordata, Tilia platophyillos, Echinacea pallida, Echinacea purpurea, Echinacea angustufolia, Helychrisum italicum, Salvia officinalis, or Grindelia robusta.

21. The method according to claim 18, wherein said resins are derived from one or more of: Grindelia robusta, Commiphota myrrha, Boswelia serrata, Pinus mugo, Pinus sylvestris, or propolis.

22. The method according to claim 18, wherein said saponins are derived from one or more of: Grindelia robusta, Verbascum densiflorum, Glycyrrhiza glabra, Marrubium vulgare, or Poligala senega.

23. The method according to claim 18, wherein said honey is bee honey and/or honeydew honey.

24. The method according to claim 18, wherein said antioxidants are polyphenols selected from the group consisting of: flavonoids, phenols, tannins, flavonoids, and mixtures thereof; and/or vitamins.

25. The method of treatment according to claim 18, wherein said mixture consists of:
polysaccharides of plant origin between 0.1 and 0.25% by weight of said composition,
antioxidants of plant origin between 0.009 and 0.05% by weight of said composition,
resins of vegetable origin between 0.01 and 0.05% by weight of said composition,
saponins of plant origin between 0.0003 and 0.001% by weight of said composition,
honey between 20 and 35% by weight of said composition, and
further glucose, sucrose, and/or fructose between 45 and 55% by weight of said composition.

26. The method according to claim 18, wherein said mixture consists of:
polysaccharides of plant origin between 0.2 and 0.7% by weight of said composition,
antioxidants of plant origin between 0.02 and 0.1% by weight of said composition,
resins of vegetable origin between 0.04 and 0.1% by weight of said composition,
saponins of plant origin between 0.0009 and 0.002% by weight of said composition, and
honey between 75 and 85% by weight of said composition.

27. The method according to claim 18, wherein said mixture consists of:
polysaccharides of plant origin between the 1.6 and 3% by weight of said composition;
antioxidants of plant origin between 0.1 and 0.8% by weight of said composition;
resins of vegetable origin between 0.1 and 0.8% by weight of said composition;
saponins of plant origin between 0.006 and 0.02% by weight of said composition;
honey between 3 and 5% by weight of said composition;
glucose, sucrose, and/or fructose between 75 and 85% by weight of said composition; and
inulin between 1.5 and 2.5% by weight of said composition.

28. The method according to claim 18, wherein said mixture consists of:
polysaccharides of plant origin between the first, second 2.5% by weight of said composition;
antioxidants of plant origin between 0.1 and 0.6% by weight of said composition;
resins of vegetable origin between 0.3 and 1% by weight of said composition;
saponins of plant origin between 0.007 and 0.02% by weight of said composition;
honey between 5 and 8% by weight of said composition, and
further glucose, sucrose, fructose between 70 and 80% by weight of said composition and
inulin between 3.5 and 4.5% by weight of said composition.

29. The method according to claim 18 further comprising one or more selected from the group consisting of: pharmaceutically acceptable excipient, humectant, carrier, binding, diluting, disintegrating, lubricant, gliding, preservative, aggregating, flavoring, and anti-adherent agent.

30. The method according to claim 29, wherein said preservative agents when present are one or more selected from the group consisting of: benzoates, parabens, and sorbates; said aggregating agents when present are one or more selected from the group consisting of: gums, natural and synthetic polymers, and sugars; said flavoring agents when present are one or more selected from the group consisting of: natural lemon flavor, sweet orange natural flavor, eucalyptus natural flavor, myrtle natural flavor, and peach natural flavor; said humectant agents when present are one or more selected from the group consisting of: sodium lauryl sulphate and sorbitan esters; said carrier agents when present are one or more selected from the group consisting of: water, demineralized water, and deionized water; said binding agents when present are one or more selected from the group consisting of: natural gums, tragacanth, gum arabic, animal gelatin, and polyvinylpyrrolidone; said diluting agents when present are one or more selected from the group consisting of: sugar, sugar alcohols, maltodextrin, and inorganic salts; said disintegrating agents when present are one or more selected from the group consisting of: rice starch, corn starch, and potato starch; said lubricant agents when present are one or more selected from the group consisting of: magnesium stearate and polyethylene glycols; said gliding agents when present are colloidal silica; and said anti-adherent agents when present are talc.

31. The method according to claim 18, wherein said persistent cough is a cough characterized by a duration of more than 10 days or more than two weeks or more than three weeks.

32. The method according to claim 18, wherein said persistent cough is a cough associated with inflammation of the upper respiratory tract (URTI) and/or to post-nasal drip.

33. The method according to claim 18, wherein said treatment is carried out on an adult patients, patient of adolescent age, geriatric patient, pregnant patient, or pediatric patient.

34. The method according to claim 18, wherein said composition is in the form of a capsule, tablet, lozenge, granules, powder, syrup, elixir, hard gelatine, soft gelatine, suspension, emulsion, or solution.
